(19) United States
(12) Patent Application Publication
(10) Pub. No.: US 2021/0214729 A1
(43) Pub. Date: Jul. 15, 2021

(54) SUBSTITUTION OF THE MESSENGER RNA CAP WITH TWO RNA SEQUENCES INTRODUCED AT THE 5-PRIME END THEREOF

(71) Applicant: MESSENGER BIOPHARMA, RIOM (FR)

(72) Inventor: Jerome LEMOINE, RIOM (FR)

(21) Appl. No.: 17/055,061

(22) PCT Filed: May 15, 2019
(86) PCT No.: PCT/FR2019/051103

§ 371 Date: Nov. 12, 2020

(30) Foreign Application Priority Data
May 15, 2018 (FR) 1854052

(51) Int. Cl. C12N 15/115; C12N 7/00; C07K 14/005; A61P 37/04

(52) U.S. Cl. CPC C12N 15/115; C12N 7/00; C07K 14/005; C12N 2770/32271; C12N 2310/16; C12N 2310/3513; C12N 2770/32222; A61P 37/04

(57) ABSTRACT

The invention relates to a messenger ribonucleic acid (mRNA) molecule lacking a cap molecule, for which the cost of in vitro transcription synthesis is greatly reduced, comprising, from 5′ to 3′, at least one copy of a GUCAGRYC(N₇₋₁₉)GCCA(N₁₂₋₁₉)UGCNRYCUG consensus sequence which is resistant to the Xrn1 exoribonuclease, a copy of an internal ribosome entry site (IRES) RNA sequence, and an open reading frame.

### FIELD

The present invention relates to the field of ribonucleic acids, and more particularly to the in vitro synthesis of messenger ribonucleic acid (mRNA), its stability and its translation into a polypeptide, in particular in transfected cells.

### INTRODUCTION

mRNA is an essential molecule in the production of polypeptides on an industrial scale. Its stability and the efficiency of its transcription and translation strongly influence the downstream yield of polypeptides and, therefore, their cost.

mRNA is also a molecule of choice in pharmaceutical compositions used in gene therapy and genetic vaccination. Indeed, the integration of an mRNA molecule into the genome of a transfected cell has never been demonstrated, in contrast to DNA molecules. However, it has poor stability in solution as it is sensitive to degradation by ribonucleases. The synthesis of RNA molecules that are stable in vitro and in vivo is therefore critical to reducing the quantities of mRNA required for optimum therapeutic efficacy and thus reducing their cost. In addition, the use of smaller amounts of more stable mRNA reduces the risk of treatment-related side effects.

In vivo synthesis occurs in cultured cells, such as yeasts or bacteria. However, this method has many disadvantages. The purification of intact mRNA of interest is expensive and complex, in particular due to degradation problems and the presence of other cellular RNAs. It generally generates a lower yield than that obtained by in vitro transcription.

As a result, the commonly used large-scale mRNA synthesis process is a cell-free in vitro transcription system. This method uses only a few purified compounds (i.e. a reaction buffer, a DNA molecule carrying a gene, a recombinant RNA polymerase and four ribonucleotide triphosphates), and the only RNA to be produced is the mRNA that one wishes to synthesize. At the end of the reaction, there are no mRNA degradation products owing to the absence of RNase in the reaction mixture. There are also no other RNA species, which is a major advantage over mRNA synthesis by cultured cells. The purification of mRNA is thus greatly simplified.

To ensure its stability in eukaryotic cells, an mRNA has a cap molecule at its 5′ end, which protects the latter from exoribonucleases. The cap is a complex molecule made up of two guanosines linked by their 5′ carbon by a chain of three phosphate groups. The terminal guanosine is methylated at position 7 of the guanine. The mRNA is stabilized by the cap's resistance to progressive 5′ to 3′ enzymatic degradation carried out by the Xrn1 exoribonuclease. In addition to its role in stabilizing mRNA, the cap performs other functions, including ribosome recruitment (Cowling, 2010). To do so, the cap begins by recruiting translation initiation factors which in turn recruit ribosomes. The latter then translate mRNA into proteins.

On an industrial scale, to improve mRNA stability and enable efficient translation of mRNA into polypeptides in transfected cells, a cap molecule should be incorporated at the 5′ end. During in vitro transcription, the cap molecule may be added during a step subsequent to transcription using a capping enzyme, such as the 2′-O-methyltransferase from Vaccina virus (Martin et al., 1975). However, this additional step increases the complexity of synthesis, requires purification of the capping enzyme, and is not very efficient (Contreas et al., 1982). In addition, this enzyme requires S-adenosyl-L-methionine, which is an unstable molecule in aqueous solution.

To simplify in vitro transcription, cap analogs have been developed in the prior art, which, however, are not satisfactory. As an example, the P¹-(5′-7-methyl-guanosyl) P³-(5′-(guanosyl))triphosphate) analog or the P¹-(5′-2,2,7-trimethyl-guanosyl) P³-(5′-(guanosyl))triphosphate) analog can be cited. These analogs allow for co-transcriptional capping mediated by phage RNA polymerase, thereby avoiding the additional step of cap synthesis, and may also improve mRNA stability. However, depending on the type of analog, up to 50% of the molecules incorporated have a reversed orientation, with the 7-methylguanosine nucleotide being adjacent to the RNA molecule rather than in terminal position, thus reducing the stability and efficiency of mRNA translation (Pasquinelli et al., 1995). “Anti-reverse” type cap analogs (ARCA) have since been developed, such as that described in U.S. Pat. No. 7,074,596. These analogs prevent reverse incorporation of the molecule. However, they remain unsatisfactory as their use induces a substantial decrease in the yield of in vitro mRNA synthesis. In addition, synthesis of the cap molecule and its many chemically modified analogs is expensive due to the complexity of these compounds. The inclusion of one of these molecules in the in vitro transcription reaction mixture on an industrial scale greatly increases the cost of mRNA synthesis.

Indeed, to ensure a high capping efficiency during in vitro transcription, it is necessary to provide cap analog in excess, thus contributing to the high cost of raw materials. Indeed, a ratio of 4 cap analog molecules is recommended per GTP molecule to maximize the chances that each mRNA molecule has a cap. Despite this, it is estimated that only 80% of synthesized mRNA will be capped. In addition, GTP concentration is reduced by 5-fold as compared to the other three ribonucleotide triphosphates, which decreases transcription yield by 5-fold.

One way to reduce the high cost of in vitro transcription is to reduce the cost of producing the DNA and/or RNA polymerase used in this method. However, the cost reductions obtained remain relatively small. Alternatively, it is possible to synthesize a circular uncapped mRNA by in vitro transcription in a cell-free system (WO 2014/186334 A1). However, the authors demonstrated that circular mRNA is less efficiently translated in a transfected cell than a capped linear mRNA (see e.g. FIGS. 2 and 3 and paragraphs [00121] and [00131] of WO 2014/186334 A1).

As a result, there is still a need for an mRNA molecule having high stability as well as high translational efficiency, preferably having stability and translational efficiency at least as high as a capped mRNA molecule. There is also a need for an RNA molecule which has a simple production method and for which the manufacturing cost is significantly reduced.

### DESCRIPTION

The present invention relates to a stable uncapped mRNA molecule which can be translated with high efficiency. This mRNA is particularly advantageous as its production cost is much lower than that of a conventional mRNA comprising a cap molecule or an analog thereof. The present invention also relates to an mRNA molecule whose in vitro transcription yield is improved as compared to that of a conventional mRNA comprising a cap molecule or an analog thereof. Indeed, the mRNA of the invention is also advantageous because, despite the significant reduction in its production cost and/or the increase in its synthesis yield, said mRNA is at least as efficient as a conventional capped mRNA when transfecting cultured cells and tissues. Indeed, the inventors have very surprisingly demonstrated that the levels and durations of expression obtained after in vivo transfection are at least as high as those of a capped mRNA. In particular, expression kinetics of a reporter protein in Caco-2 cells are identical whether transfected with the mRNA of the invention or a control capped mRNA (FIGS. 2 and 3). Likewise, the inventors have very surprisingly demonstrated that the expression of a reporter protein is 2-fold to about 10-fold greater when the mRNA of the invention is transfected in vivo, in the dermis or the muscle of a mouse, than when a control capped mRNA is transfected (FIG. 5 and FIG. 15). However, the reaction is simplified because the additional capping step is not necessary. There is also no need to include a cap analog molecule in the reaction mixture during in vitro transcription. The mRNA molecule of the invention is therefore clearly advantageous in terms of reduced costs and facilitated production as compared to an mRNA of the prior art, for an expression that is at least as high.

For the purposes of the present application, the term “mRNA molecule” means any linear chain of ribonucleotides. In the present application, these sequences are expressed in the 5′ to 3′ direction starting with the 5′-UTR region.

By “ribonucleotide” is meant any natural ribonucleotide (e.g. guanine, cytidine, uridine, adenosine), as well as analogs of these nucleotides as well as nucleotides having chemically or biologically modified bases (e.g. by methylation, alkylation, acylation, thiolation, etc.), intercalated bases, modified ribose groups and/or modified phosphate groups.

The inventors have surprisingly demonstrated herein that the cap at the 5′ end of an mRNA molecule can be replaced by at least one copy of a sequence resistant to the Xrn1 exoribonuclease (xrRNA), derived from the 3′-UTR region of a virus of the Flavivirus genus, preferably accompanied by an internal ribosome entry site (IRES) by open reading frame. Very advantageously, the production cost of such an mRNA molecule by in vitro transcription is reduced by about 30-fold as compared to a capped mRNA molecule, and its yield is improved. In addition, the mRNA molecule of the invention is particularly stable in transfected cells, while being efficiently translated, even though it is uncapped.

By “cap” is meant the 7-methylguanosine (N7-methyl guanosine or m7G) nucleoside as well as any mutant, variant, analog or fragment thereof which can be linked to the first nucleotide transcribed from an mRNA by a 5′-5′ triphosphate bond. Without limitation, cap analogs include non-methylated analogs (e.g. P¹-(Guanosyl) P³-(5′-(guanosyl)) triphosphate), monomethylated analogs (e.g. P¹-(5′-7-methyl-guanosyl) P³-(5′-(guanosyl)) triphosphate), trimethylated analogs (e.g. P¹-(5′-2,2,7-trimethyl-guanosyl) P³-(5′-(guanosyl)) triphosphate), or analogs having a substitution of the 3′-OH group of the m⁷ guanine moiety with a 3-O-methyl group (e.g. the ARCA P¹-(5′-(3′-O-methyl)-7-methyl-guanosyl) P³-(5′-(guanosyl))triphosphate).

According to a first aspect, the invention therefore relates to a messenger ribonucleic acid (mRNA) molecule lacking a cap molecule comprising:
a 5′-UTR region comprising at least one copy of a GUCAGRYC(N₇₋₁₉)GCCA(N₁₂₋₁₉)UGCNRYCUG (xrRNA) consensus sequence,
at least one copy of an internal ribosome entry site (IRES) RNA sequence; and
at least one open reading frame.

Preferably, an IRES RNA sequence is located upstream of each open reading frame.

According to a preferred embodiment, the invention relates to a messenger ribonucleic acid (mRNA) molecule lacking a cap molecule comprising from 5′ to 3′:
a 5′-UTR region comprising at least one copy of a GUCAGRYC(N₇₋₁₉)GCCA(N₁₂₋₁₉)UGCNRYCUG (xrRNA) consensus sequence,
a copy of an internal ribosome entry site (IRES) RNA sequence; and
an open reading frame.

Preferably, the mRNA molecule also comprises a 3′-UTR region.

Preferably, the mRNA molecule also comprises at least one RNA aptamer which promotes penetration of the mRNA molecule into cells, preferably into muscle cells. The RNA aptamer can notably promote penetration directly or indirectly via a peptide.

Preferably, the mRNA molecule further comprises a stem-loop at the 5′ end.

In a preferred embodiment, the invention relates to a messenger ribonucleic acid (mRNA) molecule lacking a cap molecule consisting of, from 5′ to 3′:
a 5′-UTR region comprising at least one copy of a GUCAGRYC(N₇₋₁₉)GCCA(N₁₂₋₁₉)UGCNRYCUG (xrRNA) consensus sequence,
a copy of an internal ribosome entry site (IRES) RNA sequence; and
an open reading frame.

It has been demonstrated in the prior art that the 3′-UTR untranslated region of Flavivirus has the property of blocking Xrn1 and thus protecting the downstream sequence (Chapman et al., 2014). This region of RNA is composed of at least one sequence that folds on itself forming a complex three-dimensional structure, which inhibits the progression of Xrn1 and thus the degradation of viral subgenomic RNAs. However, such sequences have never before been inserted in 5′ of an mRNA. Indeed, it was feared that these sequences may need to be in the context of the 3′-UTR of the Flavivirus genome to perform their function. In particular, these three-dimensional structures may not fold correctly when no longer surrounded by sequences of the flaviviral 3′-UTR region.

While the prior art suggests that the xrRNA sequences inhibit translation, the inventors have surprisingly demonstrated here that the protective and translation-initiating functions of the cap can be successfully replaced by at least one copy of an xrRNA sequence and at least one copy of an IRES sequence, preferably from the EMCV virus, without affecting translation efficiency. Surprisingly, the use of xrRNA and IRES sequences even allows for dramatically improved translation efficiency.

By “Flavivirus” is meant any virus of the Flavivirus genus, including yellow fever, dengue, West Nile (WNV), Zika, Japanese encephalitis, Rocio, Murray Valley, Bagaza virus, Kokobera, Ntaya, Kedougou, Sepik, Saint Louis, Usutu, Alfuy, Wesselbron, Ilheus, Bussuquara, Tembusu, Chaoyang, Yokose, Donngang virus as well as any other virus of the Flavivirus genus.

The invention thus relates to a stable mRNA molecule comprising at least one copy of an xrRNA sequence in the 5′ region of said mRNA. Said mRNA is efficiently translated into protein at levels and for durations similar to that of a capped molecule. According to a preferred embodiment of the invention, the mRNA molecule comprises two copies of xrRNA.

By “RNA sequence resistant to Xrn1” or “xrRNA” is meant any polynucleotide sequence making it possible to reduce, slow or prevent the degradation of an mRNA by the Xrn1 exoribonuclease. Preferably, the xrRNA sequence comprises a consensus sequence.

By “consensus sequence” is meant any sequence comprising at least the sequence represented by: 5′-GUCAGRYC(N₇₋₁₉)GCCA(N₁₂₋₁₉)UGCNRYCUG-3′, wherein each N may independently represent a nucleotide selected from A, C, T, G, and U or an analog thereof. Each R represents a purine and each Y represents a pyrimidine. The number of N nucleotides between the conserved bases can vary in the consensus sequence, from 5′ to 3′, from 7 to 19 bases and from 12 to 19 bases, as indicated. Said sequence forms a three-dimensional structure of the stem-loop type.

Preferably, the mRNA molecule comprises at least one copy of an xrRNA sequence selected from the sequences represented by: SEQ ID NO: 1 to SEQ ID NO: 44. When the mRNA molecule comprises more than one copy of said sequences, those copies may be the same or different. Thus, more preferably, the mRNA molecule comprises at least one copy of one of the sequences represented by SEQ ID NO: 11 and SEQ ID NO: 26. Even more preferably, the mRNA molecule comprises one copy of the sequence represented by SEQ ID NO: 11 and one copy of the sequence represented by SEQ ID NO: 26.

Preferably, when there are two xrRNA sequences in the 5′-UTR region, they are separated by a spacer sequence. Likewise, there may be a spacer sequence between an xrRNA sequence and an IRES sequence. Preferably, the spacer sequence between two xrRNA sequences corresponds to SEQ ID NO: 47. Preferably, the spacer sequence between the xrRNA sequence and the IRES sequence corresponds to SEQ ID NO: 48. A spacer sequence can also be present between two open reading frames or between an open reading frame and an IRES sequence, when the mRNA molecule comprises at least two open reading frames.

By “spacer sequence” is meant any non-coding polynucleotide sequence that makes it possible to physically separate the sequence upstream from said spacer sequence from the sequence downstream. The mRNA molecule according to the invention can notably comprise one or more spacer sequences.

In some embodiments, the spacer sequence can be from 2 to 300 nucleotides in length. Preferably, it is between 2 and 10 nucleotides, even more preferably between 2 and 5 nucleotides. Alternatively, it may be between 10 and 150 nucleotides and, even more preferably, between 15 and 40 nucleotides. Preferably, the spacer sequence does not generate secondary structures.

The mRNA of the invention further comprises at least one internal ribosome entry site (IRES) RNA sequence.

By “internal ribosome entry site” or “IRES” is meant any polynucleotide sequence which allows translation of an mRNA molecule to be initiated independently of the cap. Such sequences interact directly with translation initiation factors, which then recruit ribosomes at a translation initiation codon. Many IRES sequences are known (Mokrejs et al., 2010). The person skilled in the art will therefore be able to identify sequences functioning as IRES' and choose those suitable for implementation of the invention. The IRES sequence according to the invention can thus be of eukaryotic or viral origin, e.g. from the Picornavirus genus. Preferably, it is derived from the encephalomyocarditis virus (EMCV) (Borman et al. 1995) or from human eIF4G mRNA. Even more preferably, the DNA sequence of the IRES corresponds to SEQ ID NO: 45.

IRES sequences are usually located in the 5′-UTR region. They may also be located between two open reading frames, which makes it possible to initiate bi- or polycistronic translation from a single mRNA. In a preferred embodiment, the mRNA of the invention comprises a single copy of an IRES sequence in the 5′ region of said mRNA. According to a preferred embodiment, a second copy of an IRES sequence is located between two open reading frames. According to yet another preferred embodiment, the mRNA of the invention comprises one copy of an IRES sequence in the 5′ region and one copy of an IRES sequence between two open reading frames. Advantageously, said IRES sequence(s) is/are located downstream of the xrRNA sequence(s). This organization advantageously makes it possible to protect these sequences from the Xrn1 exoribonuclease. When the mRNA molecule comprises at least two IRES sequences, said sequences may be the same or different. In particular, one or more IRES sequences may be selected according to their efficiency in initiating translation. The selection of two different IRES sequences is particularly advantageous when the mRNA molecule comprises at least two different open reading frames and the translation efficiency desired for the first open reading frame differs from that desired for the second.

IRES elements also have complex three-dimensional structures. Therefore, reciprocal interference between xrRNA and IRES sequences is possible via the formation of pairings between RNA sequences from each of the two regions. Such interference could prevent the formation of the correct structures of the xrRNA and IRES sequences, respectively. In addition, xrRNA structures could notably prevent recruitment of translation initiation factors and ribosomes by the IRES. Even more unexpectedly, the inventors have shown that the presence of the xrRNA and IRES sequences makes it possible to obtain protein expression yields in transfected cells which are at least similar to those obtained with a capped mRNA molecule. The xrRNA and IRES sequences together can therefore replace a cap or cap analog molecule. They are essential to ensure the translation of an open reading frame contained in the mRNA of the invention and mRNA stability.

In transfected cells, the mRNA of the invention is thus at least as stable as a capped mRNA, while also being at least as efficiently translated. In addition, the cost of its synthesis by in vitro transcription is greatly reduced when compared to that of a capped mRNA.

According to a particular embodiment, the mRNA of the invention further comprises a stem-loop in the 5′-UTR region upstream of the consensus sequence GUCAGRYC(N₇₋₁₉)GCCA(N₁₂₋₁₉)UGCNRYCUG (xrRNA), preferably at the 5′ end of the mRNA molecule.

By “stem-loop” is meant any polynucleotide sequence forming a double helix-shaped structure in which the 5′ end of one strand is physically linked to the 3′ end of the other strand through an unpaired loop. Thus, the stem-loop is composed of a double-stranded stem and an unpaired single-stranded loop. Said physical bond may be either covalent or non-covalent. Preferably, said physical bond is a covalent bond. The size of the RNA loop may be e.g. between 3 and 30 nucleotides. The size of the loop is preferably at least 3 nucleotides, preferably at least 4 nucleotides. The length of the double-stranded stem may be e.g. between 5 and 50 nucleotides. The length of the stem is preferably between 5 and 50, 5 and 40, 5 and 30, 5 and 25, or more preferably between 5 and 10 nucleotides. Even more preferably, the length of the stem is 6, 7, or 8 nucleotides.

In the context of the present invention, a stem-loop is preferably formed at the 5′ end of the mRNA molecule. According to a preferred embodiment, the stem-loop has the sequence of SEQ ID NO: 87. The stem-loop is preferably separated from the xrRNA sequence by a spacer sequence. Preferably, said spacer sequence has a length that is equal to or less than 5 nucleotides (i.e. 5, 4, 3, or 2 nucleotides). Indeed, the inventors have very surprisingly demonstrated that the addition of a stem-loop structure at the 5′ end of the mRNA molecule (also called 5′-SL here, for stem-loop at the 5′ end) makes it possible to further improve in vivo translation, when it is proximal to the xrRNA sequence (e.g. at 5 nucleotides or less). Indeed, the inventors did not observe an advantageous effect when the stem-loop placed at the 5′ end was separated from the xrRNA sequence by a spacer sequence having a length of approximately 70 nucleotides (see FIG. 15).

Without being bound by theory, and very surprisingly, one can suppose that the xrRNA sequence masks the 5′ end from phosphatases and Xrn1, at least when this sequence is in the form of a stem-loop and in close proximity.

In a preferred embodiment, the invention therefore relates to a messenger ribonucleic acid (mRNA) molecule lacking a cap molecule comprising from 5′ to 3′:
a 5′-UTR region comprising at least one copy of a GUCAGRYC(N₇₋₁₉)GCCA(N₁₂₋₁₉)UGCNRYCUG (xrRNA) consensus sequence,
a copy of an internal ribosome entry site (IRES) RNA sequence; and
an open reading frame,

further comprising a stem-loop at the 5′ end.

The mRNA molecule may further comprise a second IRES sequence followed by a second open reading frame. The mRNA molecule may further comprise an RNA aptamer as defined herein, located between the stem-loop and the xrRNA sequence(s) or, preferably, between the xrRNA sequence(s) and the IRES sequence. The mRNA molecule may further comprise a 3′-UTR region as defined herein.

In a preferred embodiment, the invention relates to a messenger ribonucleic acid (mRNA) molecule lacking a cap molecule comprising from 5′ to 3′:
a 5′-UTR region comprising a stem-loop at the 5′ end followed by at least one copy of a GUCAGRYC(N₇₋₁₉)GCCA(N₁₂₋₁₉)UGCNRYCUG (xrRNA) consensus sequence,
optionally, an RNA aptamer;
a copy of an internal ribosome entry site (IRES) RNA sequence; and
an open reading frame.

In a preferred embodiment, the invention relates to a messenger ribonucleic acid (mRNA) molecule lacking a cap molecule consisting of, from 5′ to 3′:
a 5′-UTR region comprising a stem-loop at the 5′ end followed by at least one copy of a GUCAGRYC(N₇₋₁₉)GCCA(N₁₂₋₁₉)UGCNRYCUG (xrRNA) consensus sequence,
optionally, an RNA aptamer;
a copy of an internal ribosome entry site (IRES) RNA sequence; and
an open reading frame; and
a 3′-UTR region.

By “aptamer” is meant any nucleic acid having properties of recognition and specificity associated with its ability to adopt particular three-dimensional structures, in particular similar to monoclonal antibodies (see e.g. Dunn et al., 2017). An aptamer may be composed of DNA, RNA and/or modified RNA, preferably RNA. Said aptamer may be composed of 6 to 50 nucleotides, such as ribonucleotides as defined above. As a non-limiting example, the aptamer may be isolated according to various techniques which are well-known to the person skilled in the art, such as the in vitro identification of an aptamer by one or more in vitro selection cycles or from combinatorial libraries of a large number of compounds of random sequence by an iterative selection method (“SELEX” technique). The in vitro identification of an aptamer by selection advantageously makes it possible to obtain aptamers having a precise effect or function, without however needing to know the target against which said aptamer is directed. The manufacture or selection of aptamers is described e.g. in European patent application EP0533838. Advantageously, RNA aptamers have been identified in the context of the present invention according to their ability to penetrate cells of interest, more particularly tissue cells, preferably muscle cells (e.g. muscle fiber cells). Advantageously, the aptamer according to the invention is capable of crossing a cell membrane, more preferably the plasma membrane and/or the endosomal membrane of a mammalian cell.

The aptamer according to the invention preferably comprises 6 to 50 nucleotides, more preferably from 10 to 45 nucleotides, from 20 to 40 nucleotides, even more preferably from 30 to 40 nucleotides. Preferably, the aptamer is composed of ribonucleotides, such as those as defined above. Preferably, the aptamer according to the invention has at least 70% identity, more preferably at least 80% identity, at least 90% identity, at least 95% identity, at least 96% identity, at least 97% identity, at least 98% identity, even more preferably at least 99% identity with aptamer A having the sequence of SEQ ID NO: 64, aptamer B having the sequence of SEQ ID NO: 65, or aptamer C having the sequence of SEQ ID NO: 66.

The percentages of identity to which reference is made in the context of the description of the present invention are determined on the basis of a global alignment of the sequences to be compared, that is to say on an alignment of the sequences taken in their entirety over the entire length using any algorithm that is well-known to the person skilled in the art such as the algorithm of Needleman and Wunsch, 1970. This sequence comparison may be performed using any software well-known to the person skilled in the art, e.g. Needle software using the “Gap open” parameter equal to 10.0, the “Gap extend” parameter equal to 0.5 and a Blosum 62 matrix.

Preferably, the aptamer according to the invention is chosen from aptamer A having the sequence of SEQ ID NO: 64, aptamer B having the sequence of SEQ ID NO: 65, and aptamer C having the sequence of SEQ ID NO: 66, even more preferably, chosen from aptamer A having the sequence of SEQ ID NO: 64 and aptamer C having the sequence of SEQ ID NO: 66.

Preferably, the mRNA molecule according to the invention comprises at least one copy of an aptamer capable of crossing a membrane, preferably a mammalian plasma membrane and/or endosomal membrane. Thus advantageously, when the RNA molecule according to the present invention comprises an aptamer, said aptamer promotes its penetration into cells, preferably into muscle or skin cells. Preferably, the mRNA molecule according to the invention comprises at least one copy of aptamer A having the sequence of SEQ ID NO: 64, aptamer B having the sequence of SEQ ID NO: 65, and/or aptamer C having the sequence of SEQ ID NO: 66.

The aptamer promoting penetration of the mRNA molecule into target cells does not necessarily need to be protected from degradation by exonucleases, which predominantly occurs in the cytosol of cells. Thus, according to a particular embodiment, the mRNA molecule comprises the aptamer in the 5′-UTR region upstream of one or more GUCAGRYC(N₇₋₁₉)GCCA(N₁₂₋₁₉)UGCNRYCUG (xrRNA) consensus sequence(s). According to an alternative embodiment, the aptamer is placed in the 5′-UTR region downstream of one or more GUCAGRYC(N₇₋₁₉)GCCA(N₁₂₋₁₉)UGCNRYCUG (xrRNA) consensus sequence(s) but upstream from IRES sequences and open reading frames. (See FIGS. 1J and 1K for representative diagrams of these two possibilities.) Thus, according to a preferred embodiment, the invention relates to a messenger ribonucleic acid (mRNA) molecule lacking a cap molecule comprising from 5′ to 3′:
a 5′-UTR region comprising at least one aptamer capable of crossing a cell membrane, preferably a plasma and/or endosomal membrane, preferably of a mammalian cell, preferably at least one aptamer selected from aptamers A, B, and C as described herein, and at least one copy of a GUCAGRYC(N₇₋₁₉)GCCA(N₁₂₋₁₉)UGCNRYCUG (xrRNA) consensus sequence,
a copy of an internal ribosome entry site (IRES) RNA sequence; and
an open reading frame.

Preferably, the mRNA molecule comprises the aptamer in the 5′-UTR region upstream of the GUCAGRYC(N₇₋₁₉)GCCA(N₁₂₋₁₉)UGCNRYCUG (xrRNA) consensus sequence(s) (see e.g. FIG. 1F). However, the RNA molecule may also comprise the aptamer downstream of the GUCAGRYC(N₇₋₁₉)GCCA(N₁₂₋₁₉)UGCNRYCUG (xrRNA) consensus sequence(s), e.g., between the xrRNA consensus sequence(s) and the IRES sequence(s).

Thus, according to a preferred embodiment, the invention relates to an mRNA molecule lacking a cap molecule comprising from 5′ to 3′:
a 5′-UTR region comprising a stem-loop at the 5′ end followed by at least one copy of a GUCAGRYC(N₇₋₁₉)GCCA(N₁₂₋₁₉)UGCNRYCUG (xrRNA) consensus sequence,
at least one aptamer capable of crossing a cell membrane;
a copy of an internal ribosome entry site (IRES) RNA sequence; and
an open reading frame.

While aptamers B and C were selected for their ability to penetrate muscle fiber cells, aptamer A corresponds to the “shot47” aptamer, identified by Tsuji et al., 2013. Aptamer A advantageously binds to a polyhistidine-type peptide motif with high affinity. Aptamer A can thus bind any molecule (e.g. a peptide or a protein) comprising a polyhistidine tag (e.g. the HHHHHH motif). As a non-limiting example, the mRNA molecule according to the invention can be linked via aptamer A to a molecule allowing it to better penetrate cells, such as a “cell penetrating peptide” or “CPP”, said CPP comprising a poly-histidine tag.

By “cell penetrating peptide” or “CPP” is meant any peptide, polypeptide, or protein which is capable of crossing a cell membrane, more preferably the plasma membrane and the endosomal membrane, of a mammalian cell. Advantageously, the CPP retains this property when it is linked to another molecule, in particular an mRNA molecule, thus causing the latter to cross the membrane. In the context of the present invention, any possible mechanism of membrane crossing is contemplated including, e.g., energy-dependent transport mechanisms (i.e. active, e.g. endocytosis) and energy independent transport mechanisms (e.g. diffusion). In general, CPPs are cationic peptides (Poillot and De Waard, 2011). As a non-limiting example, the mRNA molecule may be non-covalently bound, due to its negative charge, to a CPP, taking advantage of electrostatic interactions and/or hydrophobicity. Alternatively, the mRNA molecule may be covalently bound to a CPP. CPPs may form oligomers consisting of at least two identical or different peptide molecules. In the context of the present invention, a CPP preferably non-covalently binds to an RNA aptamer. Indeed, this type of bond is advantageous given its simplicity, by simple mixing of the CPP and mRNA molecules, its low cost and the fact that these molecules are entirely biodegradable. Indeed, no non-natural and non-biodegradable chemical group is necessary.

The length of the CPPs according to the present invention is preferably from about 8 amino acid residues to about 60 amino acid residues. More preferably, the length is from 8 to 40 amino acid residues, more preferably from 8 to 30 amino acid residues, even more preferably from 10 to 25 amino acid residues (e.g., 13 or 20 amino acid residues). The person skilled in the art will recognize, however, that the length of the CPPs is not necessarily limited to those described above. Derivatives of the CPPs described herein, having e.g. different lengths, can in particular be created by the skilled person in view of their general knowledge.

As a non-limiting example, the CPP of the invention may be the “M12” CPP as described by Gao X. et al., 2014, the “CPP2” or “CPP3” CPP as described by Kamada et al., 2007, or the CPP “CPP1” as described by Lee et al., 2012, as well as any variant or derivative of these. According to a preferred embodiment, said CPP comprises a polyhistidine motif (e.g. hexahistidine), preferably linked to said CPP by a spacer. Preferably, the spacer is a hydrophilic spacer, advantageously unstructured and uncharged, even more advantageously composed of glycines and serines. Preferably, the spacer is about 21 amino acids in length, preferably 21 amino acids.

Preferably, the CPPs according to the invention have at least 70% identity, more preferably at least 80% identity, at least 90% identity, at least 95% identity, at least 96% identity, at least 97% identity, at least 98% identity, even more preferably at least 99% identity with the M12-H6 peptide having the sequence of SEQ ID NO: 75, the CPP1-H6 peptide having the sequence of SEQ ID NO: 76, the CPP2-H6 peptide having the sequence of SEQ ID NO: 77, or the CPP3-H6 peptide having the sequence of SEQ ID NO: 78. According to a particularly preferred embodiment, the CPP according to the invention is selected from: M12-H6 having the sequence of SEQ ID NO: 75, CPP1-H6 having the sequence of SEQ ID NO: 76, CPP2-H6 having the sequence of SEQ ID NO: 77, and CPP3-H6 having the sequence of SEQ ID NO: 78, even more preferably selected from: CPP1-H6 having the sequence of SEQ ID NO: 76, CPP2-H6 having the sequence of SEQ ID NO: 77, and CPP3-H6 having the sequence of SEQ ID NO: 78. Advantageously, the CPP is linked non-covalently or covalently to the mRNA molecule, preferably non-covalently. Advantageously, the CPP is linked to an RNA aptamer included in the mRNA molecule (i.e. aptamer A when the CPP comprises a polyhistidine tag).

Preferably, the CPPs of the present invention do not have significant cytotoxic and/or immunogenic effects on their target cells after crossing the plasma membrane, i.e. they do not interfere with cell viability, cell transfection and/or penetration. The term “non-significant,” as used in this context, means that less than 50%, preferably less than 40% or 30%, preferably less than 20% or 10% and in particular less than 5% of target cells are killed after the mRNA molecule linked to the CPP has crossed the plasma membrane, and has therefore been internalized by the cell. The person skilled in the art is familiar with methods for determining the cytotoxicity of a given compound and/or the viability of a target cell to which such a compound is applied (see, e.g., Ausubel et al., 2001). Corresponding assay kits are commercially available from various suppliers. In specific embodiments, the potential intrinsic cytotoxic and/or immunogenic effects of a CPP of the invention may be “masked” by introducing one or more modifications in the peptide, e.g. by means of chemical synthesis or recombinant DNA. Such modifications can include, for example, the addition, removal or substitution of functional groups or varying the positions of these functional groups. The person skilled in the art is well-aware of how such “masking” can be accomplished for a given peptide.

Thus, according to a preferred embodiment, the invention relates to an mRNA molecule lacking a cap molecule comprising from 5′ to 3′:
a 5′-UTR region comprising at least the aptamer A RNA of SEQ ID NO: 64 and at least one copy of a GUCAGRYC(N₇₋₁₉)GCCA(N₁₂₋₁₉)UGCNRYCUG (xrRNA) consensus sequence,
a copy of an internal ribosome entry site (IRES) RNA sequence, and
an open reading frame,

wherein said mRNA molecule is linked to a cell penetrating peptide (CPP) fused to a polyhistidine tag, said CPP preferably being selected from: M12-H6 having the sequence of SEQ ID NO: 75, CPP1-H6 having the sequence of SEQ ID NO: 76, CPP2-H6 having the sequence of SEQ ID NO: 77, and CPP3-H6 having the sequence of SEQ ID NO: 78. Preferably, said CPP is non-covalently linked to the aptamer.

By “open reading frame” is meant any polynucleotide sequence which can be translated into a polypeptide of interest. An open reading frame is read in blocks of three successive nucleotides, called codons, with each codon representing an amino acid. During translation, the polypeptide is synthesized by translating the codons of said open reading frame by ribosomes.

The first amino acid of the polypeptide is generally indicated on the mRNA molecule by the AUG codon, which thus indicates the start of the open reading frame. Other start codons are known, such as AUN, or NUG, in which N corresponds to A, C, U or G. The end of the polypeptide is indicated on the mRNA molecule in the form of a UAA, UGA, or UAG stop codon. The stop codon indicates the end of the open reading frame on the mRNA molecule.

Open reading frames according to the invention are more particularly open reading frames whose translation in the transfected cell generates products of therapeutic or vaccinal interest for applications in human or veterinary medicine. Preferably, the products of therapeutic interest are proteins.

Among the proteins of therapeutic interest, one may more particularly refer to enzymes, blood derivatives, hormones, lymphokines: interleukins, interferons, TNF, etc. (FR 92 03120), growth factors, neurotransmitters or their precursors or synthetic enzymes, trophic factors: BDNF, CNTF, NGF, IGF, GMF, aFGF, bFGF, NT3, NT5, etc., apolipoproteins: ApoAI, ApoAIV, ApoE, etc. (FR 93 05125), dystrophin or a minidystrophin (FR 91 11947), tumor suppressor proteins: p53, Rb, Rap1A, DCC, k-rev, etc. (FR 93 04745), factors involved in coagulation: Factors VII, VIII, IX, etc., pro-apoptotic proteins: thymidine kinase, cytosine deaminase, etc., or even all or part of a natural or artificial immunoglobulin (Fab, ScFv, etc., see e.g. WO 2011/089527), an RNA ligand (WO 91/19813), etc.

The protein of interest coded by the mRNA may also be an antigen, capable of generating an immune response in humans or animals, for the realization of vaccines. They may in particular be antigenic proteins specific for the Epstein Barr virus, the HIV virus, the hepatitis B virus (EP 185 573), the pseudo-rabies virus, or even specific for tumors (EP 259 212).

Finally, the protein of interest may be an adjuvant protein, which stimulates the immune response, in order to improve the efficacy of a vaccine.

The protein of interest coded by the mRNA may be a protein which has a favorable effect on an uncapped mRNA molecule or the protein expressed by said molecule. This favorable effect could result from different mechanisms. As a non-limiting example, a protein coded by the uncapped mRNA may increase the stability of an uncapped mRNA molecule by binding to said molecule or by degrading at least one protein having RNase activity. As a non-limiting example, a protein coded by the uncapped mRNA may increase the translation of said molecule by promoting the recruitment of initiation factors or by binding capped cellular mRNAs in order to inhibit their translation. As a non-limiting example, the protein of interest coded by the mRNA is the 2Apro protein from a Picornavirus, such as human rhinovirus type 2 (HRV2). Without being bound by theory, one can suppose that the 2Apro protein increases the expression of the uncapped mRNA in view of its protease activity, cleaving the N-terminal end of the eIF4G initiation factor and thus preventing said initiation factor from recognizing a capped mRNA. This cleavage could make it possible to reduce the competition between the mRNA of the invention and capped mRNAs in vivo. Indeed, the inventors have surprisingly shown that the co-transfection of cells with a first mRNA according to the invention coding 2Apro and a second mRNA according to the invention coding a reporter protein allows expression of the reporter protein to be increased. The presence of the 2Apro protein is therefore particularly advantageous as allows the specific expression of the protein coded by an uncapped mRNA of the invention to be increased (FIG. 7).

According to a preferred embodiment, the mRNA of the invention codes a 2Apro protein, preferably a 2Apro protein from a Picornavirus, even more preferably from the HRV2 virus. According to a particular embodiment, the 2Apro protein has the sequence of SEQ ID NO: 81. According to a particular embodiment, the 2Apro protein is coded by an mRNA having the sequence of SEQ ID NO: 80.

The open reading frame may also code therapeutic mRNA. This can be, e.g., an antisense sequence, whose expression in the target cell makes it possible to control the transcription or the translation of cellular mRNAs. Such sequences can e.g. be transcribed, in the target cell, into RNAs that are complementary to cellular mRNAs and thus block their translation into protein, according to the technique described in patent EP 140 308.

Preferably, the mRNA of the invention comprises, in addition to the xrRNA sequence(s) and the IRES sequence, an open reading frame coding a polypeptide of interest. Preferably, this open reading frame is located downstream of the xrRNA sequence(s). The person skilled in the art will readily understand that the mRNA can include several open reading frames. Thus, the mRNA may be monocistronic, bicistronic or polycistronic. The mRNA is monocistronic when it only has a single open reading frame. It is bicistronic when it includes two open reading frames and polycistronic when it includes at least two open reading frames.

The mRNA of the invention can also include one or more non-coding regions. These non-coding regions may notably be regions between two open reading frames. In this case, IRES sequences are advantageously present in these non-coding regions located between two open reading frames.

According to a particular embodiment, the messenger ribonucleic acid (mRNA) molecule lacking a cap or a cap analog molecule comprises from 5′ to 3′:
a 5′-UTR region comprising at least one copy of a GUCAGRYC(N₇₋₁₉)GCCA(N₁₂₋₁₉)UGCNRYCUG (xrRNA) consensus sequence, the copy(ies) of said sequence being followed by a single copy of an internal ribosome entry site (IRES) RNA sequence;
an open reading frame; and
a 3′-UTR region comprising a poly(A) sequence.

Advantageously, said mRNA molecule also comprises at least one RNA aptamer which promotes the penetration of said molecule into target cells, preferably muscle cells. Advantageously, said mRNA molecule comprises at least one aptamer chosen from aptamer A having the sequence of SEQ ID NO: 64, aptamer B having the sequence of SEQ ID NO: 65, and aptamer C having the sequence of SEQ ID NO: 66.

According to another particular embodiment, the messenger ribonucleic acid (mRNA) molecule lacking a cap or cap analog molecule consists of 5′ to 3′:
a 5′-UTR region comprising at least one copy of a GUCAGRYC(N₇₋₁₉)GCCA(N₁₂₋₁₉)UGCNRYCUG (xrRNA) consensus sequence, the copy(ies) of said sequence being followed by a single copy of an internal ribosome entry site (IRES) RNA sequence;
an open reading frame; and
a 3′-UTR region comprising a poly(A) sequence.

By “5′-UTR region” is meant any region of nucleic acid which is upstream of the translation initiation codon. This region is non-coding but can include elements that regulate mRNA expression downstream. In addition to the xrRNA and IRES elements, this region may include other elements such as the riboswitch and/or T-box. In some embodiments, the 5′-UTR region may be from 10 to 2000 nucleotides in length. Preferably, it is comprised between 50 and 1500 nucleotides, and more preferably from 200 to 1000 nucleotides.

By “3′-UTR region” is meant any region of nucleic acid which is located downstream of the translation termination codon. This region may influence the expression and/or stability of mRNA, its location in a cell, or contain binding sites for proteins or small interfering RNAs or microRNAs. This region may include, e.g., such elements as a polyadenylated tail (poly(A)), a histone stem-loop structure, and/or a region rich in pyrimidines or purines. It may contain coding or non-coding sequences. In some embodiments, the 3′-UTR region may be from 50 to 500 nucleotides in length. Preferably, it is comprised of between 50 and 200 nucleotides, and more preferably from 50 to 100 nucleotides. In a preferred embodiment, the mRNA comprises a polyadenylated tail (poly(A)), comprising a nucleotide sequence of adenine or an analog or variant thereof, of 10 to 300 nucleotides, and preferably between 50 and 100 nucleotides.

According to a second aspect, the invention relates to a deoxyribonucleic acid (DNA) molecule comprising a polynucleotide which can be transcribed into the mRNA molecule of the invention. Preferably, the DNA molecule comprises the 5′-UTR region of SEQ ID NO: 50, 71, 72, 73, 85, or 86.

Preferably, said DNA molecule is comprised in an expression cassette.

By “expression cassette” is meant herein a DNA fragment comprising a polynucleotide of interest, for example a polynucleotide which can be transcribed into the mRNA molecule of the invention, operably linked to one or more regulatory elements controlling the expression of gene sequences, such as, e.g., promoter sequences and enhancer sequences.

A polynucleotide is “operably linked” to regulatory elements when these different nucleic acid sequences are combined on a single nucleic acid fragment in such a way that the function of one is affected by the others. For example, a regulatory DNA sequence is “operably linked” to a DNA sequence coding an RNA or a protein if the two sequences are situated in such a way that the regulatory DNA sequence affects the expression of the DNA coding sequence (in other words, the DNA coding sequence is under the transcriptional control of the promoter). The coding sequences may be operably linked to regulatory sequences in a sense orientation as well as an antisense orientation. Preferably, the coding sequences of the invention are operably linked to regulatory sequences in the sense orientation.

By “regulatory sequences” or “regulatory elements” is meant herein polynucleotide sequences that are necessary to affect the expression and maturation of the coding sequences to which they are ligated. Such regulatory sequences notably include transcription initiation and termination sequences, promoter sequences and enhancer sequences; signals of efficient RNA maturation, such as splicing and polyadenylation signals; sequences stabilizing cytoplasmic mRNAs; sequences improving translational efficiency (e.g., Kozak sequences); sequences which increase protein stability; and, if necessary, sequences which increase protein secretion.

Preferably, the regulatory sequences of the invention comprise promoter sequences, i.e. the gene coding the mRNA of the invention is preferably operably linked to a promoter which allows for the expression of said corresponding mRNA. A gene coding the mRNA of the invention is preferably operably linked to a promoter when it is located downstream of the latter, that is to say 3′ thereof, thereby forming an expression cassette.

The term “promoter” as used herein refers to a nucleotide sequence, most often located upstream (5′) of the coding sequence, which is recognized by the RNA polymerase and other factors necessary for transcription, and thereby controls the expression of said coding sequence. A “promoter” as used herein includes in particular minimal promoters, i.e. short DNA sequences composed of a TATA box and other sequences which allow the transcription start site to be specified. A “promoter” within the meaning of the invention also comprises nucleotide sequences including a minimal promoter and regulatory elements capable of controlling the expression of a coding sequence. For example, the promoter sequences of the invention may contain regulatory sequences such as enhancer sequences which can influence the level of expression of a gene.

Advantageously, the promoters according to the invention are those which function with an RNA polymerase used in a cell-free transcription system. For example, promoters recognized by the RNA polymerases of SP6 and T7 phages are widely known to the person skilled in the art. Thus, the pMBx-luc2 vectors which carry promoters recognized by T7 RNA polymerase (Rogé and Betton, 2005) were used in the experimental section below. Vectors containing such promoters are, moreover, commercially available.

In a particular embodiment, the invention comprises a DNA molecule coding the mRNA of the invention which is operably linked to at least one regulatory element. Preferably, the DNA molecule coding the mRNA of the invention is operably linked to a promoter sequence, located upstream, thereby forming an expression cassette. In another embodiment the invention consists of a DNA molecule coding the mRNA of the invention which is operably linked to a promoter sequence, located upstream, thereby forming an expression cassette.

Even more preferably, the DNA molecule of the invention comprises:
a promoter recognized by T7 RNA polymerase, said promoter comprising a sequence represented by SEQ ID NO: 46;
a 5′-UTR region comprising a sequence represented by SEQ ID NO: 50, 71, 72, 73, 85, or 86;
an open reading frame; and
a 3′-UTR region comprising a sequence chosen from the sequences represented by SEQ ID NOs: 53, 54, 55, and 56.

Advantageously, the regulatory sequences of the invention comprise transcription terminator sequences, that is to say that the gene coding the mRNA of the invention is preferably operably linked to a transcription terminator. The term “transcription terminator” herein designates a genome sequence which marks the end of transcription of a gene or an operon, into messenger RNA. The mechanism of transcription termination differs between prokaryotes and eukaryotes. The person skilled in the art knows the signals to use according to the different cell types. For example, if they wish to express the mRNA of the invention in a bacterium, they will use a Rho-independent terminator (inverted repeat sequence followed by a series of Ts (uracils in the transcribed RNA) or a Rho-dependent terminator (constituted of a consensus sequence recognized by the Rho protein). A gene coding the mRNA of the invention is preferably operably linked to a terminator when it is located upstream of the latter, that is to say 5′ thereof, thereby forming an expression cassette.

Advantageously, the terminators according to the invention are those which function with an RNA polymerase used in a cell-free transcription system. For example, terminators recognized by RNA polymerases of SP6 and T7 phages are widely known to the person skilled in the art. Vectors containing such terminators are commercially available.

In a particular embodiment, the invention comprises a DNA molecule coding the mRNA of the invention which is operably linked to at least one regulatory element and at least one transcription terminator.

In a third aspect, the invention also relates to a vector comprising at least the DNA or mRNA molecule according to the invention.

The term “vector” as used herein refers to a nucleic acid molecule capable of transporting another nucleic acid to which it has been linked. One type of vector is a “plasmid,” which refers to a circular double loop of single-stranded DNA into which additional DNA segments can be ligated. Another type of vector is a viral vector, in which additional DNA segments can be ligated into the viral genome. Alternatively, the viral vector may comprise mRNA in a viral genome (such as, e.g., retroviruses or RNA viruses). Certain vectors are capable of autonomous replication in a host cell into which they are introduced (e.g., bacterial vectors having a bacterial origin of replication and episomal mammalian vectors). Other vectors (such as e.g., integrative mammalian vectors) may be integrated into the genome of a host cell upon introduction in the host cell, and are thus replicated with the host genome.

Numerous vectors into which one can insert a nucleic acid molecule of interest in order to introduce and maintain it in a eukaryotic or prokaryotic host cell, are known to the person skilled in the art. The choice of an appropriate vector depends on the intended use for this vector (e.g. replication of the sequence of interest, expression of this sequence, maintenance of this sequence in extrachromosomal form or even integration into the chromosomal material of the host), as well as the nature of the host cell (e.g., plasmids are preferably introduced in bacterial cells, while YACs are preferably used in yeasts). These expression vectors may be plasmids, YACs, cosmids, retroviruses, episomes derived from EBV, and any vectors that a person skilled in the art may deem appropriate for the expression of said sequences. In a preferred embodiment of the invention, the vector used to code the mRNA of the invention is a vector capable of being propagated in bacteria. More preferably, this plasmid comprises a promoter recognized by an RNA polymerase used in a cell-free transcription system, such as those of the SP6 and T7 phages. Even more preferably, this promoter carried by this plasmid is capable of directing the expression of the mRNA of the invention in the presence of at least said RNA polymerase.

Preferably, the vector of the invention comprises an origin of replication to allow the multiplication of said vector in the host cell. The term “origin of replication” (also called ori) is a unique DNA sequence allowing the initiation of replication. It is from this sequence that unidirectional or bidirectional replication begins. The person skilled in the art knows that the structure of the origin of replication varies from one species to another; it is therefore specific, though they all have certain characteristics. A protein complex is formed at this sequence and allows for DNA opening and the start of replication.

Advantageously, the vector containing the expression cassette of the invention further comprises a selection marker, in order to facilitate the identification of cells containing said vector, in particular after transformation. A “selection marker” according to the invention is a polynucleotide sequence carried by a vector, which allows for the identification and selection of cells having said vector. Selection markers are well-known to the person skilled in the art. Preferably, it is a gene coding a protein conferring antibiotic resistance.

The vectors of the invention, comprising the nucleic acid(s) of interest of the invention, are prepared by methods commonly used by the person skilled in the art. The resulting clones may be introduced into an appropriate host by standard methods known to the person skilled in the art for introducing polynucleotides into a host cell. Such methods may be transformation using dextran, precipitation with calcium phosphate, transfection using polybrene, protoplast fusion, electroporation, encapsulation of polynucleotides in liposomes, biolistic injection and direct microinjection of DNA into the nucleus. It is also possible to associate said DNA or mRNA sequence (isolated or inserted into a plasmid or viral vector) with a substance allowing it to cross the membrane of host cells, such as a transporter such as a nanotransporter or a preparation of liposomes, or cationic polymers. In addition, these methods can advantageously be combined, e.g. by using electroporation associated with liposomes.

According to a preferred embodiment of the invention, the vector of the invention comprises a DNA molecule coding the mRNA of the invention. Preferably, the vector of the invention is a plasmid. Even more preferably, the vector of the invention comprises an expression cassette, a transcription terminator, an origin of replication, and a selection marker. In another preferred embodiment, the vector consists of an expression cassette.

According to another preferred embodiment of the invention, the vector comprises an mRNA molecule of the invention. Preferably, the vector of the invention is a virus or a synthetic RNA vector.

According to another aspect, the invention relates to a host cell comprising said vector. The term “host cell,” as used herein, is intended to refer to a cell in which a recombinant expression vector has been introduced in order to express the mRNA of the invention. This term is to be understood to encompass not only said particular host cell but also the progeny thereof. It is understood that certain modifications can occur over generations due to mutations or environmental influences. Progeny may, as a result, not be exactly identical to the parent cell, but are nevertheless also included in the term “host cell” as used herein.

The DNA and/or vectors described above are particularly useful for producing large amounts of the mRNA of the invention.

According to another aspect, the invention relates to a method for producing the mRNA of the invention using the DNA or the vector described above. The mRNA can thus be produced by any method known to the person skilled in the art. This can be done, e.g., by chemical synthesis, by in vivo expression or by in vitro expression.

Preferably, the mRNA is expressed in vitro, using an acellular mRNA expression system. By “cell-free mRNA expression system” is meant in the context of the invention a biochemical system allowing for the synthesis of the mRNA of the invention in the absence of a cell. The cell-free system is based on the use of the transcriptional machinery of an organism to produce a specific mRNA from exogenous genetic information. The cell-free mRNA expression system in the context of the invention therefore contains all the elements necessary for the production of mRNA in the absence of a cell. The organisms from which this machinery is extracted are many and varied, and come from prokaryotic and eukaryotic organisms.

In particular, this system comprises, inter alia, the transcriptional machinery originating from the cell. More particularly, this system comprises an RNA polymerase capable of recognizing the promoter of the expression cassette described above. In the presence of appropriate nucleotides and under the appropriate ionic conditions, this RNA polymerase is thus capable of directing the transcription of the gene coding the mRNA of the invention. Such systems have been well-known to the person skilled in the art for several decades (for a review, see e.g. Beckert and Masquida, 2011). Many methods are available for transcribing DNA into RNA in cell-free systems. It is also possible to use kits offered by many companies: New England Biolabs, Sigma Aldrich, Thermo Scientific, Promega, Roche Diagnostics, Ambion, Invitrogen, etc.

In addition to RNA polymerase, the cell-free in vitro transcription system includes a reaction buffer. Advantageously, said system comprises each of the four ribonucleotide triphosphates. Very advantageously, these four ribonucleotide triphosphates are present at identical concentrations. In particular, the concentration of GTP is identical to that of the other three ribonucleotide triphosphates. The yield of in vitro transcription of the mRNA of the invention is thus much higher than that of a capped mRNA resulting from an in vitro reaction, which greatly reduces the cost of mRNA synthesis.

Preferably, the production method comprises a step of purifying said mRNA.

In a preferred embodiment, plasmid DNA, comprising a promoter recognized by a phage RNA polymerase and followed by a DNA sequence coding the mRNA of interest, is contacted with a phage RNA polymerase in a cell-free in vitro transcription system. The mRNA synthesized by said method can then be purified. Preferably, said plasmid DNA is linearized before being contacted with RNA polymerase in the cell-free in vitro transcription system. More preferably, said DNA is linearized by enzymatic digestion downstream of the 3′-UTR region. Even more preferably, said DNA is linearized by enzymatic digestion with Ssp1 or Eco53kl.

Preferably, the RNA polymerase is bacteriophage T7 RNA polymerase.

The mRNA molecule is capable of directing the production of a polypeptide of interest in a eukaryotic organism into which it is introduced. Therefore, it is particularly suitable for gene therapy or genetic vaccination. The mRNA molecule of the present invention can thus be used as a drug or as a vaccine.

According to another aspect, the invention also relates to a pharmaceutical composition or a vaccine composition.

More particularly, the present invention thus relates to a pharmaceutical or vaccine composition comprising the mRNA of the invention. The mRNAs included in the composition transfect cells, which can then translate them into proteins. Preferably, these proteins have prophylactic activity or therapeutic activity.

Indeed, the inventors have surprisingly shown that the mRNA according to the invention is more effective than a conventional capped mRNA during tissue transfection. Indeed, the inventors have very surprisingly shown that the levels and durations of expression obtained after in vivo transfection are at least as high as those of a capped mRNA. In particular, the in vivo expression of the mRNA of the invention, in the dermis or muscle of a mouse, is greater than that of a control capped mRNA (FIG. 5, FIG. 15). In addition, the inventors have very surprisingly shown that the mRNA according to the invention persists in cells over the long term (i.e. more than 7 weeks). Finally, the inventors have surprisingly shown that the co-transfection of cells with a first mRNA according to the invention coding the 2Apro protein and a second mRNA according to the invention coding a second protein makes it possible to increase the translation of this second protein.

Thus, according to another aspect of the invention, the pharmaceutical composition comprises the 2Apro protein or an mRNA coding said protein. Preferably, the 2Apro protein has the sequence of SEQ ID NO: 81. Preferably, the mRNA coding said protein has the sequence of SEQ ID NO: 80. According to a particular embodiment of the invention, the pharmaceutical composition comprises at least two different molecules of uncapped messenger ribonucleic acid (mRNA) comprising from 5′ to 3′:
a 5′-UTR region comprising at least one copy of a GUCAGRYC(N₇₋₁₉)GCCA(N₁₂₋₁₉)UGCNRYCUG (xrRNA) consensus sequence,
a copy of an internal ribosome entry site (IRES) RNA sequence; and
an open reading frame,

wherein at least one of the mRNA molecules comprises an open reading frame coding the 2Apro protein.

Preferably, said pharmaceutical composition comprises a first mRNA molecule, said first mRNA molecule comprising a reading frame coding the 2Apro protein, and at least a second mRNA molecule, said second mRNA molecule comprising an open reading frame coding a second protein of interest. More preferably, said second protein of interest is not the 2Apro protein. Even more preferably, said second molecule of interest is an antigen or a therapeutic protein such as defined above. According to a particular embodiment of the invention, the molar ratio between the first mRNA and the second mRNA is comprised between 540:1 and 240:1, preferably between 540:1 and 315:1, even more preferably 465:1.

Preferably, said composition will be supplemented with an excipient and/or a pharmaceutically acceptable vehicle. In the present description, the term pharmaceutically acceptable vehicle refers to a compound or a combination of compounds that is included in a pharmaceutical composition that does not cause side effects and which allows for e.g. improved ease of administration of the active compound(s), its increased lifespan and/or effectiveness in the body, its increased solubility in solution or even improved storage. These pharmaceutically acceptable vehicles are well-known and will be adapted by the person skilled in the art according to the nature and mode of administration of the selected active compound(s). In the present description, the term “pharmaceutically acceptable excipient” refers to a compound or a combination of compounds that is included in a pharmaceutical composition that does not cause side effects and which allows for, e.g., improved ease of administration of the active compound(s), its increased lifespan and/or effectiveness in the organism, its increased solubility in solution or even improved storage. Said excipient can notably be added to the composition just before administration, e.g. if the RNA is stored in the form of a lyophilizate. Pharmaceutically acceptable excipients/vehicles are well-known and will be adapted by the person skilled in the art according to the nature and mode of administration of the selected active compound(s). Various excipients are notably described in The Science and Practice of Pharmacy by Remington, 22^{nd} ed., Pharmaceutical Press, London, UK (2013). As an example, the pharmaceutically acceptable composition comprises sterile water and/or chloroquine as an excipient. The “chloroquine” excipient also includes any variant or analog thereof, such as primaquine.

Indeed, the inventors have surprisingly shown that, under certain conditions, the co-injection of chloroquine with the mRNA makes it possible to improve transfection efficiency. In particular, transfection efficiency is improved for an mRNA complexed with a “CPP” type peptide (cf. FIG. 10).

According to a preferred embodiment, the pharmaceutical composition further comprises chloroquine. As a non-limiting example, the pharmaceutical composition comprises a weight ratio comprised between 1:0.5 to 1:4 of mRNA:chloroquine, and more particularly a weight ratio of 1:1 of mRNA:chloroquine. As a non-limiting example, the pharmaceutical composition further comprises 5 μg RNA:2.5-20 μg chloroquine.

According to a preferred embodiment, the pharmaceutical composition further comprises at least one CPP as described herein, non-covalently linked to the mRNA molecule according to the invention. Advantageously, the CPP is selected to promote penetration of the mRNA into the target cells (e.g. depending on the type of organ or cell line, e.g. muscle or dermal cells).

Preferably, these compositions will be administered by the intramuscular, intradermal, intraperitoneal or subcutaneous route, by the respiratory route, or by the topical route. These compositions are preferably intended for injection into mammalian tissues, even more preferably humans. These compositions are preferably intended to be injected by the intramuscular, intravenous, intradermal, intraperitoneal or subcutaneous route, according to methods known to the person skilled in the art. The pharmaceutical composition of the invention may be administered several times, staggered over time. Its mode of administration, dosage and optimal galenic form may be determined according to the criteria generally taken into account in establishing a treatment adapted to a patient such as e.g. the age or the body weight of the patient, the severity of their general condition, tolerance to treatment, and the side effects observed.

Parenterally administrable forms include aqueous suspensions, isotonic saline solutions, or sterile and injectable solutions which may contain pharmacologically compatible dispersing and/or wetting agents. Forms which may be administered by the respiratory route include aerosols. Topically administrable forms include patches, gels, creams, ointments, lotions, sprays, eye drops.

Methods for preparing parenterally administrable compounds will be known or obvious to the person skilled in the art and are described in more detail in, e.g., Pharmaceutical Sciences of Remington, 17th ed., Mack Publishing Company, Easton, Pa. (1985), and the 18th and 19th editions thereof. The use of media and agents for pharmaceutically active substances is well-known in the art. To obtain a pharmaceutically acceptable composition suitable for administration, said composition will comprise a sufficient amount of mRNA molecules to be therapeutically effective.

The effective dose of a compound of the invention varies as a function of numerous parameters such as, e.g., the chosen route of administration, weight, age, sex, the state of advancement of the pathology to be treated, and the sensitivity of the individual to be treated.

According to a particular aspect, the invention relates to said composition for its use in gene therapy. The composition of the present invention may be used in the treatment or modulation of a variety of diseases, e.g.: cancer, genetic diseases (such as hemophilia, thalassemia, adenosine deaminase deficiency, alpha-1 antitrypsin deficiency), diabetes, brain disorders such as Alzheimer's and Parkinson's disease, allergies, autoimmune diseases, and cardiovascular diseases.

According to another aspect, the invention relates to said composition for its use in genetic vaccination. As an example, the composition of the present invention can be used in vaccination against cancer and influenza, as well as other viral and bacterial pathogens. Vaccination may concern humans as well as pets and livestock.

The inventors have notably shown that the mRNA of the present invention is stable in vivo. Indeed, the mRNA of the invention induces the synthesis of a quantity of protein in vivo that is at least as high as that of a capped mRNA, indicating that its resistance to Xrn1 is at least as efficient as that of a capped mRNA. In addition, the inventors have notably shown that the mRNA of the present invention persists in cells over the long term. Such an mRNA molecule is also very advantageous as its production cost is much lower than that of capped mRNA molecules. Finally, the inventors have shown that mRNA comprising an RNA aptamer that directly penetrates cells (e.g. RNA aptamer C) or via a CPP peptide (e.g. RNA aptamer A), transfected cells more efficiently, which advantageously improves the production of one or more proteins of interest.

The invention will be more particularly described by means of the examples below.

### FIGURES

FIG. 1: Schema of messenger RNAs.

(A) The capped MB5-luc2 mRNA corresponds to a traditional messenger RNA coding luciferase, with a cap analog at its 5′ end and a 3′-UTR region comprising a poly(A) tail. (B) The MB5-luc2 mRNA is identical to (A) except that it lacks a cap analog. (C) The MB7-luc2 mRNA lacks a cap and has a stem-loop and the internal ribosome entry site (IRES) of the EMCV virus at its 5′ end. (D) The MB8-luc2 mRNA lacks a cap and has both a stem-loop, two Xrn1 resistant sequences (xrRNA1 and xrRNA2) from West Nile virus (WNV) and the IRES from the EMCV virus in the 5′ UTR region. (E) The MB9-luc2 mRNA is similar to (D), except that the 3′-UTR and poly(A) of the latter have been replaced by the 3′-UTR from the Kunjin virus (KUN). (F) The uncapped MB11-luc2 mRNA is similar to (D), except that aptamer A has been added in the 5′ region, upstream of the two Xrn1 resistant sequences (xrRNA1 and xrRNA2) from West Nile virus (WNV) and the IRES of the EMCV virus and that it does not have a stem-loop. The MB13-luc2 (G) and MB14-luc2 (H) mRNAs have this same configuration, but comprise a 5′ stem-loop followed by the RNA aptamers B and C, respectively. (I) The capped MB15-luc2 mRNA is similar to (A), except that the aptamer A has been added in the 5′ region after the cap. (J) The MB17-luc2 mRNA is similar to (D), except the aptamer A has been added 5′ between the stem-loop and the xrRNA sequences. (K) The MB18-luc2 mRNA is similar to (J), except that the A aptamer is placed in the 5′ region downstream of the xrRNA sequences and upstream of the IRES sequence.

FIG. 2: Luciferase expression kinetics in Caco-2 cells over four days.

Confluent Caco-2 cells were transfected with the MB5-luc2 capped (diamond) and uncapped (circle) mRNAs, as well as the MB7-luc2 (triangle) and MB8-luc2 (square) mRNAs. Expression kinetics were followed for four days.

FIG. 3: Luciferase expression kinetics in Caco-2 cells over ten days.

Confluent Caco-2 cells were transfected with the capped MB5-luc2 (diamond), MB8-luc2 (square) and MB9-luc2 (triangle) mRNAs. Expression kinetics were followed for ten days.

FIG. 4: Long-term luciferase expression kinetics in human mesenchymal stem cells.

Mesenchymal stem cells were transfected with 2 μg of MB8-luc2 mRNA complexed with pepMB1 peptide at a ratio of pepMB1 peptide positive charge:mRNA negative charge of about 2.2:1 per well in 48 well plates for 1 hour. Luciferase activity was then monitored for approximately 48 days.

FIG. 5: Transfection of mouse muscle and dermis.

The muscle and dermis of male BALB/cByJ mice were transfected with 10 μg and 5 μg, respectively, of uncapped MB8-luc2 mRNA or capped MB5-luc2 mRNA. Luciferase activity was measured in muscle and skin 16 hours and 18 hours, respectively, after the injections.

FIG. 6: Toxicology study of MB8-2Apro mRNA.

C2C12 cells were transfected with MB8-luc2 mRNA alone or MB8-luc2 mRNA mixed with MB8-2Apro mRNA at a ratio of 465:1 or 9:1, in order to assess the cytotoxic effect of 2Apro protein expression. Cytotoxicity was determined by measuring lactate dehydrogenase (LDH) released into the extracellular medium. LDH activity is determined by measuring the absorbance at 490 nm. Negative control: Untransfected cells or cells transfected with MB8-luc2 mRNA alone without lysis. Positive control: Untransfected cells lysed with Triton X-100.

FIG. 7: Effect of 2Apro protein on luciferase expression as a function of the MB8-luc2 mRNA:MB8-2Apro mRNA molar ratio.

C2C12 cells were transfected with MB8-luc2 mRNA in the presence of an increasing amount of MB8-2Apro mRNA. The molar ratio of MB8-luc2 mRNA:MB8-2Apro mRNA is from 540:1 to 240:1. The total amount of transfected mRNA was 750 ng per well. Luciferase activity was measured 18 hours after transfection.

FIG. 8: Luciferase expression kinetics over seven days in the presence or absence of MB8-2Apro mRNA.

Confluent C2C12 cells were transfected with MB8-luc2 mRNA alone (black line) or in combination with MB8-2Apro mRNA (gray dotted line). The molar ratio of MB8-luc2 mRNA:MB8-2Apro mRNA is 465:1. Expression kinetics, measured by the level of luciferase activity, were followed for seven days.

FIG. 9: Effect of 2Apro protein on luciferase expression of different messenger RNAs.

The effect of the 2Apro protein on luciferase expression from different messenger RNAs was evaluated. In each case, the mRNA coding luciferase was transfected alone (−) or co-transfected with a second mRNA coding the 2Apro protein (+) and having the same characteristics at the optimal molar ratio of 465:1. (1) MB8-luc2 mRNA alone; (2) co-transfection of MB8-luc2 mRNA with MB8-2Apro mRNA; (3) Capped MB5-luc2 mRNA alone; (4) co-transfection of capped MB5-luc2 mRNA with uncapped MB8-2Apro mRNA; (5) MB5-luc2 mRNA lacking a cap analog alone; (6) co-transfection of MB5-luc2 mRNA lacking a cap analog with MB8-2Apro mRNA lacking a cap analog; (7) MB7-luc2 mRNA alone; (8) co-transfection of MB7-luc2 mRNA with MB8-2Apro mRNA. Luciferase activity was measured 18 hours after transfection.

FIG. 10: Effect of different aptamers on transfection efficiency of a capped mRNA molecule in muscle.

Muscle of male BALB/cByJ mice was transfected with: 5 μg of MB8-luc2 mRNA, MB13-luc2 mRNA, MB14-luc2 mRNA, MB11-luc2 mRNA complexed with M12-H6 peptide, or of MB11-luc2 mRNA complexed with the M12-H6 peptide in the presence of 5 μg of chloroquine. Luciferase activity was measured 16 hours after the injections.

FIG. 11: Transfection efficiency of mRNA according to the molar ratio of CPP3-H6 peptide:MB11-luc2 mRNA in the dermis.

The dermis of male OF1 mice was transfected with 5.6 μg of MB11-luc2 mRNA complexed with the CPP3-H6 peptide at increasing molar ratios (molar ratios between 1:8 and 1125:1 of CPP3-H6:MB11-luc2 mRNA). Luciferase activity was measured 18 hours after the injections. The molar ratio allowing for optimal transfection was 1 CPP3-H6:4 MB11-luc2 mRNA.

FIG. 12: Efficiency of transfection of mRNA according to the molar ratio of CPP1-H6 peptide:MB11-luc2 mRNA in the dermis.

The dermis of male OF1 mice was transfected with 5.6 μg of MB11-luc2 mRNA complexed with the CPP1-H6 peptide at increasing molar ratios (molar ratios between 1:8 and 3:1 of CPP1-H6:MB11-luc2 mRNA). Luciferase activity was measured 18 hours after the injections. The molar ratio allowing for optimal transfection was 1 CPP1-H6:4 MB11-luc2 mRNA.

FIG. 13: Efficiency of transfection of mRNA according to the molar ratio of CPP2-H6 peptide:MB11-luc2 mRNA in the dermis.

The dermis of male OF1 mice was transfected with 5.6 μg of MB11-luc2 mRNA complexed with the CPP2-H6 peptide at increasing molar ratios (molar ratios between 1:4 and 2.75:1 of CPP2-H6:MB11-luc2 mRNA). Luciferase activity was measured 18 hours after the injections. The molar ratio allowing for optimal transfection was 2 CPP2-H6:1 MB11-luc2 mRNA.

FIG. 14: Effect of aptamer A on the transfection efficiency of a capped mRNA molecule in the dermis.

RNA aptamer A was inserted in the 5′-UTR of the conventional capped MB5-luc2 mRNA to generate the capped MB15-luc2 mRNA. The dermis of male OF1 mice was transfected with 5.6 μg of capped MB15-luc2 mRNA complexed with the CPP2-H6 peptide at a molar ratio of 2 CPP2-H6:1 MB15-luc2 mRNA or not, in view of the results obtained previously (see FIG. 12). Transfection was not improved with this construct, either in the presence or absence of the A aptamer or of the CPP2-H6 peptide.

FIG. 15: Effect of 5′-SL on the transfection efficiency of an mRNA molecule in the dermis.

The effect of a stem-loop (here “5-SL” having the sequence of SEQ ID NO: 87) on transfection efficiency when the stem-loop is followed by the xrRNA1 sequence, 5 nucleotides downstream (MB8-luc2 and MB18-luc2 mRNA), as compared to an mRNA lacking a stem-loop (MB11-luc2 mRNA) and an mRNA whose stem-loop is located more than 70 nucleotides upstream of xrRNA1 (MB17-luc2 mRNA). The conventional MB5-luc2 capped mRNA serves as a control for the efficacy of 5′-SL followed by two xrRNA sequences. The dermis of male OF1 mice was transfected with 5.6 μg of each of the different mRNAs and luciferase activity was measured 18 hours after the injections. Aptamer A of MB11-luc2 is not linked to a peptide, and therefore has no effect on the transfection efficiency of this mRNA.

### EXAMPLES

The invention is illustrated by the following non-limiting examples. These teachings include alternatives, modifications and equivalents, as will be appreciated by one skilled in the art.

### Example 1: Plasmid Construction

DNA sequences corresponding to 5′ non-coding sequences of SEQ ID NO: 49, SEQ ID NO: 50, or SEQ ID NO: 51 were chemically synthesized, integrated into a DNA vector and sequenced by ProteoGenix. The DNA fragments were excised by restriction enzymes. Plasmids of the pMBx-luc2 series were digested with the same restriction enzymes and the DNA fragments were integrated into these plasmids by the action of T4 DNA ligase. These plasmids contain the gene coding the luciferase enzyme (SEQ ID NO: 62), inserted downstream of the bacteriophage T7 promoter. A short non-coding 3′-UTR sequence followed by a transcriptional polyadenylation sequence, according to SEQ ID NO: 53, is located downstream of the luciferase gene. The different non-coding 5′-UTR sequences separate the promoter from the luciferase gene. The plasmids thus constructed were amplified, verified and linearized downstream of the transcriptional polyadenylation sequence by a restriction enzyme.

### Example 2: Plasmid Linearization and In Vitro Transcription of mRNAs

An Ssp1 restriction site is located immediately downstream of the poly(A) of each plasmid (cf. SEQ ID NO: 54). Ten micrograms of plasmid were digested with twenty units of the

Ssp1-HF restriction enzyme (New England Biolabs) in 1× CutSmart buffer for four hours at 37° C.

Eight μl of plasmid linearized by Ssp1-HF were then mixed with 2 μl of 10×T7 RNA polymerase reaction buffer, 2 μl each of the four nucleotide triphosphates (ATP, GTP, CTP and UTP) and 2 μl of T7 RNA polymerase solution (New England Biolabs). A cap analog was included in this reaction mixture for the synthesis of capped mRNAs. It was omitted for the synthesis of uncapped mRNAs.

Transcription took three to ten hours and was performed in a 37° C. block heater. Then, 1 μL of TURBO DNase (Thermo Fisher) was added to degrade the plasmid and the mixture was incubated at 37° C. for 15 minutes.

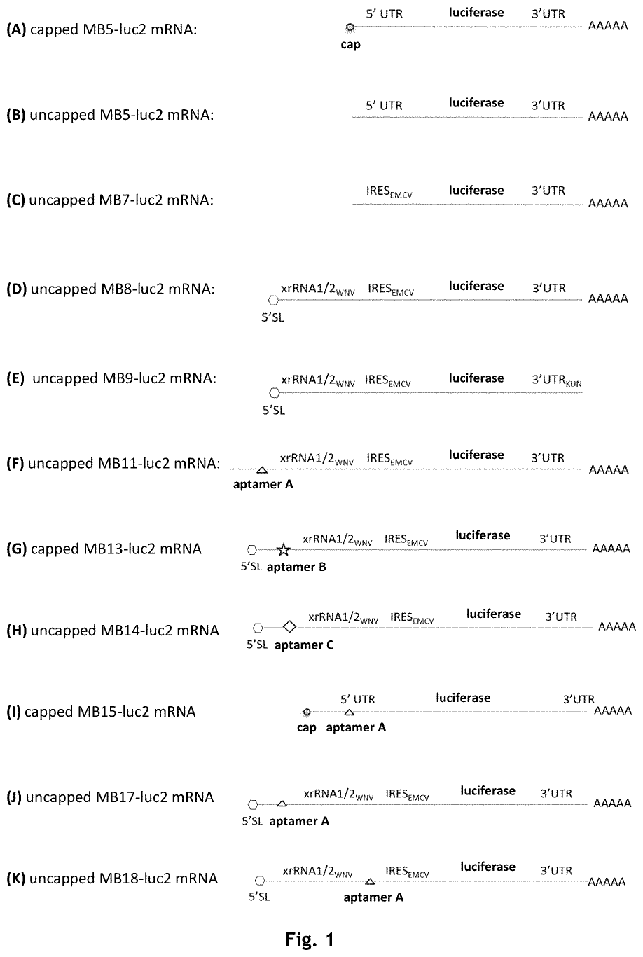

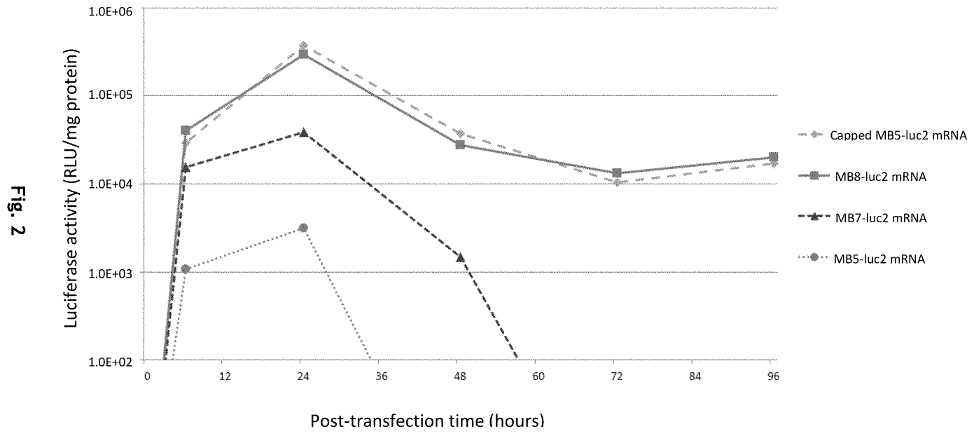

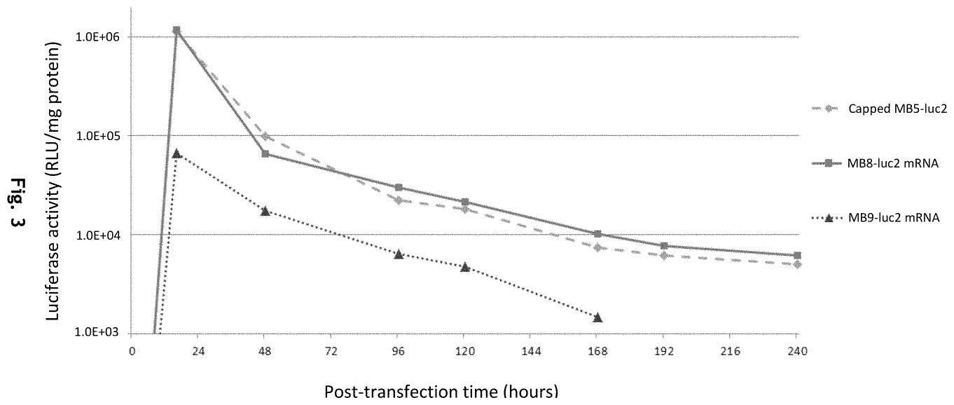

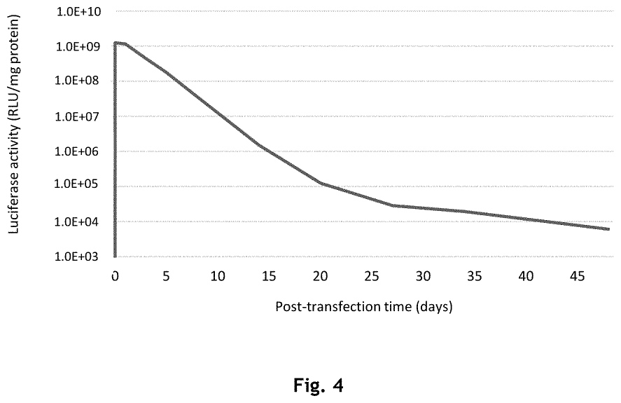

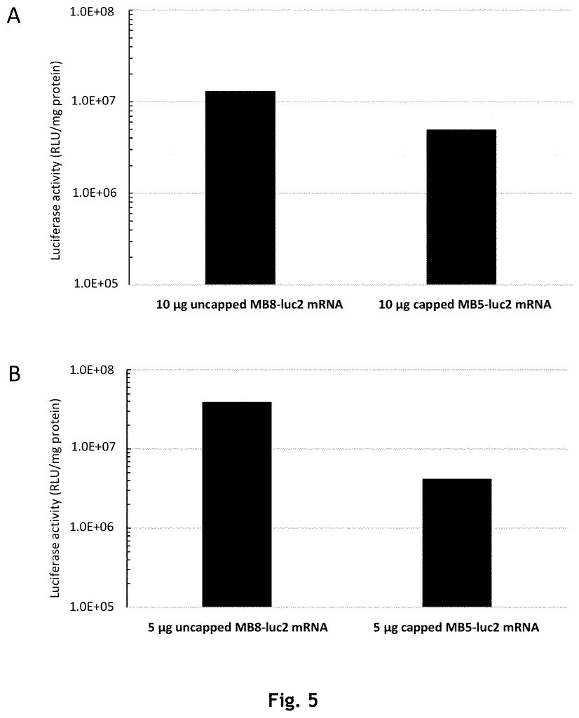

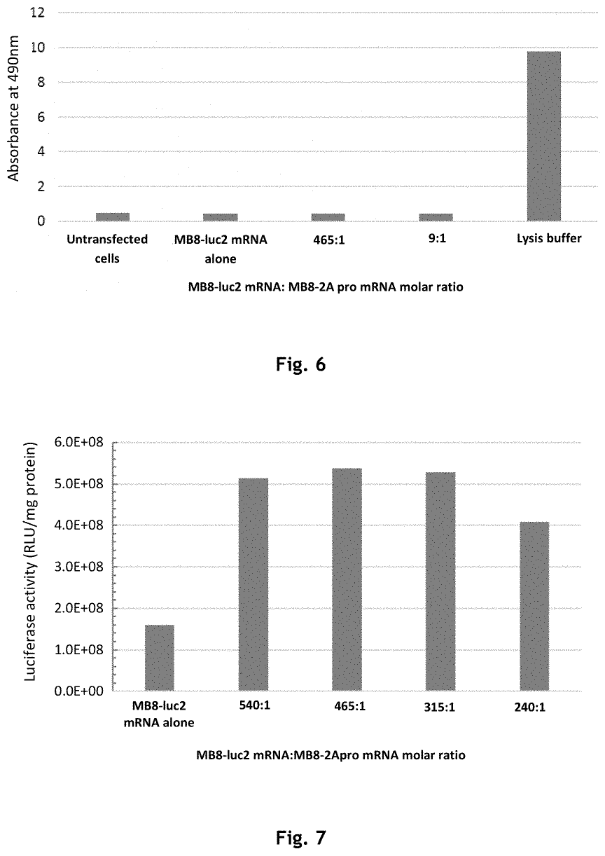

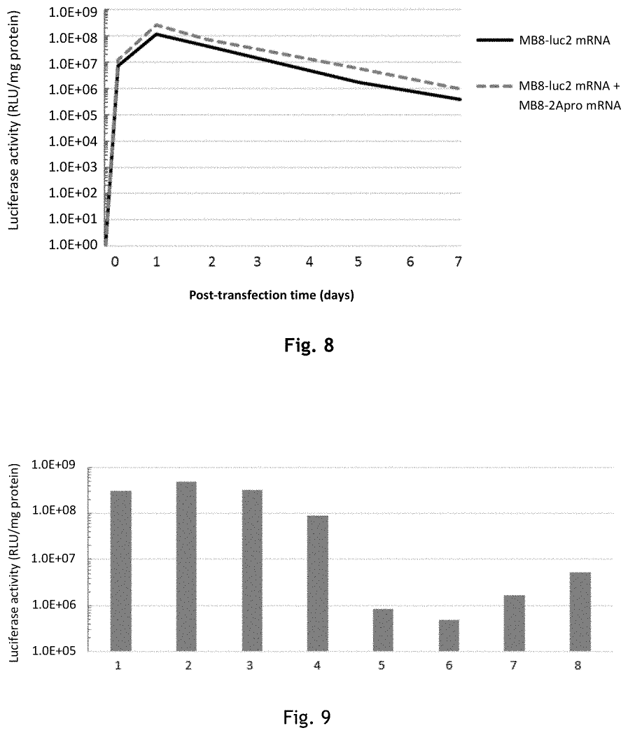

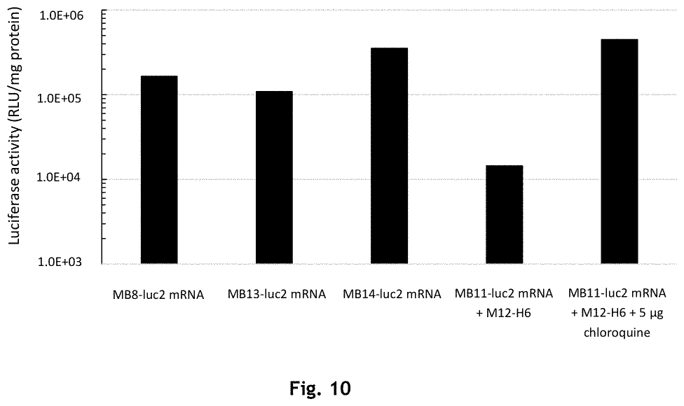

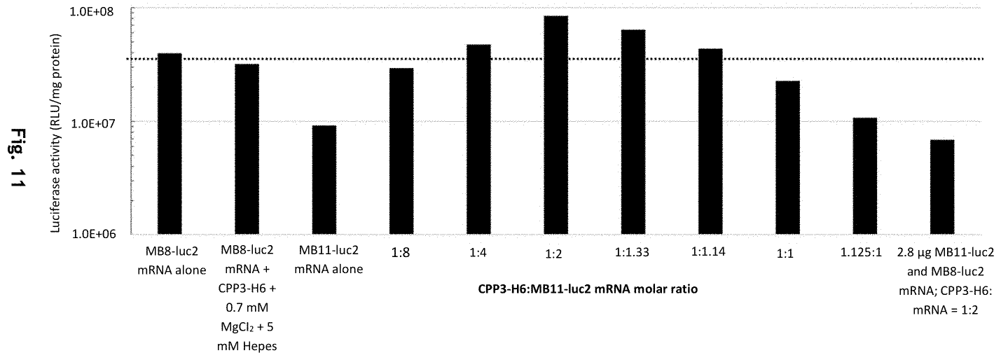

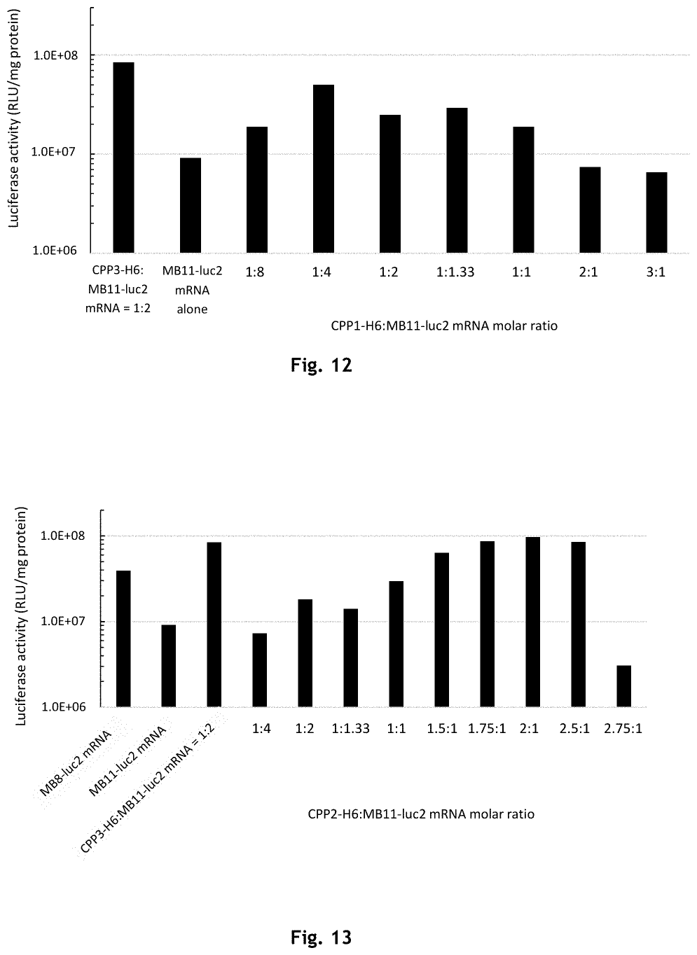

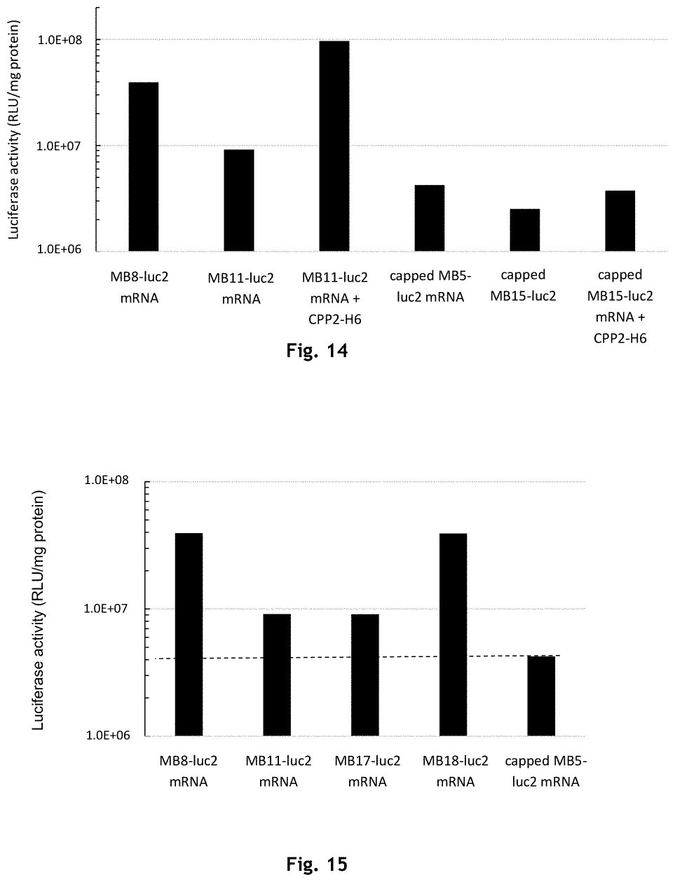

MB5-luc2 mRNA is a conventional mRNA coding luciferase. It has a 5′ non-coding sequence (UTR) according to SEQ ID NO: 57, which has been selected for optimal initiation of translation as well as a 3′-UTR region comprising a poly(A) tail according to SEQ ID NO: 60. This mRNA was synthesized with or without a cap (cf. FIG. 1 (A) and (B)).

Uncapped MB7-luc2 mRNA has at its 5′ end, in place of the 5′-UTR of the MB5-luc2 mRNA, a stem-loop at the 5′ end, followed by the IRES sequence of the EMCV virus. The latter would thus allow it to efficiently recruit ribosomes, despite the absence of a cap. However, this mRNA would be sensitive to the Xrn1 enzyme (cf. FIG. 1 (C)). The 5′-UTR region of MB7-luc2 mRNA corresponds to SEQ ID NO: 58 and the 3′-UTR region corresponds to SEQ ID NO: 60.

Uncapped MB8-luc2 mRNA has a stem-loop at its 5′ end, followed, in the 5′-UTR region, by two successive sequences resistant to Xrn1, derived from the 3′-UTR of Flavivirus WNV, called xrRNA1 and xrRNA2 (Kieft et al., 2015). These sequences are followed by that of the IRES of the EMCV virus, which is thus protected by the two sequences resistant to Xrn1 (cf. FIG. 1 (D)). The 5′-UTR region of MB8-luc2 mRNA corresponds to SEQ ID NO: 59 and the 3′-UTR region corresponds to SEQ ID NO: 60. The cost of producing uncapped MB8-luc2 mRNA is about 30 times lower than the cost of producing capped mRNA.

Uncapped MB9-luc2 mRNA differs from uncapped MB8-luc2 mRNA only by its 3′ end. Indeed, the 3′-UTR and the poly(A) sequence of MB8-luc2 have been replaced by the 3′-UTR region of the Kunjin virus. This region does not have a poly(A) sequence (cf. FIG. 1 (E)). The 3′-UTR region of MB9-luc2 mRNA corresponds to SEQ ID NO: 61.

Uncapped MB11-luc2 mRNA differs from uncapped MB8-luc2 mRNA only by the absence of a stem-loop at the 5′ end, and the presence of an aptamer in the 5′ region upstream of the two successive sequences resistant to Xrn1 (cf. FIG. 1 (F)). The MB11-luc2 mRNA comprises the A aptamer of SEQ ID NO: 64; the 5′-UTR region of the MB11-luc2 mRNA thus corresponds to SEQ ID NO: 67.

Uncapped MB13-luc2 and MB14-luc2 mRNAs differ from uncapped MB8-luc2 mRNA only by the presence of an aptamer in the 5′ region upstream of the two successive sequences resistant to Xrn1 (cf. FIG. 1 (G, H)). The MB13-luc2 mRNA comprises the B aptamer of SEQ ID NO: 65; the 5′-UTR region of the MB13-luc2 mRNA thus corresponds to SEQ ID NO: 68. The MB14-luc2 mRNA comprises the C aptamer of SEQ ID NO: 66; the 5′-UTR region of the MB14-luc2 mRNA thus corresponds to SEQ ID NO: 69.

MB15-luc2 mRNA corresponds to the capped MB5-luc2 mRNA, in which the A aptamer (SEQ ID NO: 64) has been inserted into the 5′-UTR region (cf. FIG. 1 (I)); the 5′-UTR region of the MB15-luc2 mRNA thus corresponds to SEQ ID NO: 70.

MB17-luc2 mRNA differs from uncapped MB11-luc2 mRNA only by the presence of a stem-loop at the 5′ end upstream of aptamer A (cf. FIG. 1 (J)). The MB17-luc2 mRNA comprises the A aptamer of SEQ ID NO: 64; the 5′-UTR region of the MB17-luc2 mRNA thus corresponds to SEQ ID NO: 83.

Uncapped MB18-luc2 mRNA differs from uncapped MB8-luc2 mRNA only by the presence of an aptamer in the 5′ region between the two successive sequences resistant to Xrn1 and the IRES sequence (cf. FIG. 1 (K)). The MB18-luc2 mRNA comprises the A aptamer of SEQ ID NO: 64; the 5′-UTR region of the MB18-luc2 mRNA thus corresponds to SEQ ID NO: 84.

### Example 3: Purification of Messenger RNAs

Purification of the different luciferase mRNAs was carried out using the MegaClear kit (Ambion). Seventy-nine μl of Elution Solution, 350 μl of Binding Solution Concentrate and 250 μl of 100% ethanol were added to the 21 μl of the previous mixture. These 700 μl were placed on a Filter Cartridge and centrifuged at 10,000×g for one minute. The filter retained the messenger RNA. Two washes were performed with 500 μl of Wash Solution, centrifuging at 10,000×g, for one minute. RNA was then eluted from the filter by adding 50 μl of Elution Solution twice and heating to 70° C. for ten minutes in a block heater. Elution was obtained by centrifugation at 10,000×g for one minute.

A second purification step was performed by precipitation with lithium chloride. Sixty μl of LiCl Precipitation Solution was added to the 100 μl of eluate. The mixture was cooled at −20° C. for one hour, before being centrifuged at maximum speed at four degrees, for 15 minutes. The pellet was washed with 500 μl of 70% ethanol and a final centrifugation was performed at maximum speed at four degrees, for 5 minutes. The messenger RNA pellet, air dried for a few minutes, was resuspended in sterile deionized water. The concentration of the mRNA solution was determined by measuring the absorbance at 260 nm, using a spectrophotometer.

### Example 4: Assembly of Messenger RNA/pepMB1 Complexes

The cationic peptide pepMB1 was synthesized, purified and lyophilized by ProteoGenix. Its amino acid sequence is as follows: CRRRRRRRRC. The lyophilizate was resuspended in sterile deionized water.

Five μg of luciferase mRNA was mixed with 5 μg of pepMB1 at a final RNA concentration of 20 μg/ml. Mixtures were incubated at room temperature (20-25° C.) for 15 minutes before being frozen at −80° C. mRNA/pepMB1 complexes were then lyophilized for about 20 hours.

### Example 5: Transfection of Caco-2 or C2C12 Cells

### Materials and Methods

### a) Culture and Seeding of Cells of the Caco-2 or C2C12 Line

All cell manipulations were performed under a laminar flow hood. The Caco-2 cell line (ECACC) was cultured in DMEM (Gibco) supplemented with non-essential amino acids, a mixture of antibiotics and an antimycotic, and fetal calf serum (15% final). Culture was performed at 37° C. in 75 cm² flasks (Corning).

When the number of cells needed to seed a 48-well plate (Corning) was reached, the cells were detached from the bottom of the flask using 3 ml of TryPLE Select 1× (Gibco) at 37° C., for 5 minutes. 7 ml of DMEM was added to neutralize the TryPLE Select 1×. The cells were centrifuged at 100×g, for 10 minutes at room temperature. The cell pellet was then resuspended in 10 ml of culture medium. 250 μl of this cell suspension was introduced into each well of a 48-well plate and the latter was placed in an incubator at 37° C. containing 5% CO₂.

C2C12 cells can be kept at confluence in the wells of a 48-well plate for about a dozen days after seeding. Beyond this period, these cells differentiate into intestinal epithelium, which affects mRNA translation. In contrast, human mesenchymal stem cells can be stored in confluent culture for more than 7 weeks. Mesenchymal stem cells (Millipore, Human Mesenchymal Stem Cell (Bone Marrow)) were thus cultured in 48-well plates (Corning) in a ready-to-use medium (Millipore, Mesenchymal Stem Cell Expansion Medium) for up to 48 days. For cells that will be lysed more than five days after transfection, the culture medium was changed three times per week.

### b) Transfection of Caco-2 or C2C12 Cells

For Caco-2 cells, transfection with each mRNA was performed in five different wells. Lyophilizates of mRNA/pepMB1 complexes (Proteogenix) were resuspended in 750 μl of transfection buffer (20 mM Hepes, 40 mM KCl and 100 mM trifluoroacetate).

For C2C12 cells, transfection with MB8 mRNA is carried out as indicated below. MB8-luc2 mRNA/pepMB1 complexes (Proteogenix) are assembled by incubating the mRNA in presence of the peptides at a ratio of positively-charged peptide:negatively charged mRNA of approximately 2.2 for 30 min at room temperature. The solution is then diluted with 3×DMEM to obtain a final DMEM at 1×.

In both cases, wells were emptied of the culture medium they contained in order to introduce 150 μl of RNA/pepMB1 complex solution (corresponding to 1 μg of mRNA per well for the Caco-2 cells and to 2 μg per well for C2C12 cells). Cells were incubated for 30 minutes (Caco-2 cells) or 1 hour (C2C12 cells) at 37° C. in a CO₂ incubator. The mRNA/pepMB1 complex solution was then aspirated and replaced with 250 μl of culture medium. Cells were then incubated, for 6 hours to 48 days depending on the cell type, at 37° C. in a CO₂ incubator.

### c) Lysis of Caco-2 or C2C12 Cells and Measurement of Luciferase Activity

Six hours to 48 days after transfection, cells were lysed in order to conduct expression kinetics of the luciferase protein. Culture medium was aspirated and replaced with 250 μl of lysis buffer (Luciferase Assay System, Promega). 20 μl of each cell lysate was placed in a tube suitable for the luminometer (Berthold Technologies). 100 μl of luciferase substrate (Promega) was added to the cell lysate by the luminometer. The latter then measured the amount of light emitted by the enzymatic reaction catalyzed by luciferase. Results are expressed in relative light units (RLU). The amount of luciferase protein produced by Caco-2 cells or C2C12 cells, by means of the luciferase mRNA, was normalized by assaying for total cell proteins with the 660 nm Protein Assay kit (Pierce). For this, 100 μl of cell lysate was mixed with 1.5 ml of reagent and absorbance was measured at 660 nm. A calibration range was performed using solutions of bovine serum albumin. Luciferase activity is therefore expressed in RLU per milligram of protein.

### Results

Results are illustrated in FIGS. 2, 3 and 4. Uncapped MB5-luc2 mRNA induces low and short expression of the luciferase protein in Caco-2 cells. In the absence of a cap, mRNA is rarely translated into protein and is rapidly degraded by Xrnl (cf. FIG. 2).

Uncapped MB7-luc2 mRNA gives stronger and more durable expression of the luciferase protein in Caco-2 cells than uncapped MB5-luc2 mRNA. The IRES region recruits ribosomes, but does not provide significant resistance to Xrn1 (cf. FIG. 2).

Uncapped MB8-luc2 mRNA induces luciferase expression kinetics similar to that obtained with capped MB5-luc2 mRNA (cf. FIGS. 2 and 3). This means that the addition of the two sequences resistant to Xrn1 of the WNV virus gives the mRNA resistance to Xrn1 similar to that of the MB5-luc2 mRNA cap. The MB7-luc2 mRNA, which lacks a cap and sequences resistant to Xrn1, induces luciferase expression which is intermediate between that of the uncapped MB8-luc2 mRNA and the uncapped MB5-luc2 mRNA (cf. FIG. 2).

MB9-luc2 mRNA differs from MB8-luc2 mRNA by its 3′-UTR end which lacks poly(A). The luciferase expression it induces in Caco-2 cells is notably lower and less durable than that induced by MB8-luc2 mRNA (cf. FIG. 3).

In human mesenchymal stem cells, MB8-luc2 mRNA surprisingly and advantageously induces luciferase expression which persists very long term. Indeed, even if the expression decreases over time, it remains detectable even 48 days after transfection (cf. FIG. 4).

### Example 6: Transfection of Mouse Muscle and Dermis

### Materials and Methods

### a) Animal Housing

For muscle, 8-week-old male BALB/cByJ mice, were housed in open cages with five animals per cage. The day/night cycles were managed by an automatic device (12 h day/12 h night). They were fed and had access to filtered water ad libitum. For skin, 6-week-old male OF1 mice, were housed in open cages with four animals per cage.

### b) Extemporaneous Preparation of mRNA Samples

For each intramuscular injection, 100 μl of a solution of naked mRNA containing 230 mM NaCl was prepared.

For each intradermal injection, 17 μl of a solution of naked mRNA containing 160 mM NaCl was prepared.

When a CPP-H6 peptide was used, this was mixed with mRNA and incubated for 30 minutes at room temperature in presence of 5 mM Hepes pH 7.5 and 0.7 mM MgCl₂.

### c) Intradermal and Intramuscular Injections of mRNA

Anesthesia of mice was achieved by the use of isoflurane. For intramuscular injections, analgesia was given by injection of buprenorphine. For intradermal injections, the skin on the back was shaved three to four days earlier (see Example 10 for details).

100 μl and 17 μl of mRNA solution was injected into the biceps femoris and skin, respectively. The animals were returned to their cages until the next morning.

### d) Skin and Muscle Samples

For muscle, mice were euthanized with CO₂ 16 hours after the injections. For skin, mice were anesthetized with isoflurane and euthanized by cervical dislocation 18 hours after the injections. The skin and muscle injection sites were harvested. These biopsies were washed with physiological saline, cut into fine pieces and placed in tubes containing lysis buffer (Promega). These tubes were immediately frozen in liquid nitrogen.

### e) Lysis of Cells From Skin and Muscle Biopsies

Each skin and muscle biopsy underwent three freeze/thaw cycles. Indeed, tubes containing the biopsies and lysis buffer were frozen at −80° C. for 10 minutes. Then, they were thawed in a water bath at room temperature for two minutes and mixed briefly using a vortex. Tubes were then centrifuged at 5000×g at 20° C. for 5 minutes in order to precipitate tissue debris and obtain a clarified supernatant of cell lysate.

### f) Measurement of Luciferase Activity

20 μl of each cell lysate was used for measurement of luciferase expression in each biopsy. A tube luminometer added 100 μl of luciferase substrate (Promega) to each sample and measured the amount of light emitted for 10 seconds. Results are expressed in relative light units or RLUs.

Cell lysates were then diluted 8 to 20-fold for protein assay, using the 660 nm Protein Assay kit (Pierce). 100 μl of diluted cell lysate was mixed with 1.5 ml of reagent for 6 minutes and the absorbance was measured at 660 nm. A calibration range was performed with bovine serum albumin from 0 to 750 μg/ml.

### Results

Results are shown in FIG. 5. Uncapped MB8-luc2 mRNA induces greater luciferase expression, in skeletal muscle (A) and in the skin (B), than the capped MB5-luc2 mRNA, 16 hours (for muscle) or 18 hours (for the dermis) after their injection. These results indicate that the presence of the two xrRNA sequences (here from the WNV virus) and IRES (here from EMCV) gives the mRNA a resistance to Xrn1 and a translation efficiency superior to that of the cap of the MB5-luc2 mRNA in vivo. Indeed, quite surprisingly, the transfection of 10 μg of MB8-luc2 mRNA in muscle tissue in mice results in a luciferase expression 2.6-fold higher than that obtained with the same dose of capped MB5-luc2 mRNA (cf. FIG. 5A). Likewise, transfection of 5 μg of MB8-luc2 mRNA in the skin results in a 9.3-fold higher luciferase expression than that obtained with the same dose of capped MB5-luc2 mRNA (cf. FIG. 5B).

The MB8-luc2 mRNA can therefore fully replace the capped MB5-luc2 mRNA, and would even be more advantageous.

### Example 7: Cellular Toxicity of the MB8-2Apro mRNA

### Materials and Methods

Expression of the 2Apro protein in a mammalian cell can induce toxicity to the point of causing cell death by apoptosis or necrosis (Goldstaub et al., 2000). During these processes, cells release lactate dehydrogenase (LDH) into the extracellular environment. This LDH activity can be measured using a commercial kit, CytoTox96 Non-Radioactive Cytotoxicity Assay, Promega.

The MB8-2Apro mRNA (SEQ ID NO: 80) is an mRNA coding the 2A nonstructural protein (2Apro, having the sequence of SEQ ID NO: 81) from the genome of human rhinovirus 2 (HRV2). It has a 5′ non-coding sequence (UTR) according to SEQ ID NO: 57, which is identical to that of the MB8-luc2 mRNA, as well as a 3′-UTR region comprising a poly(A) tail according to SEQ ID NO: 60.

750 ng of MB8-luc2 mRNA alone or of a mixture of MB8-luc2 mRNA and MB8-2Apro mRNA were complexed with the pepMB1 peptide, as described in Example 4.

MB8-luc2 mRNA alone or MB8-luc2 mRNA mixed with MB8-2Apro mRNA, at two different molar ratios, were transfected into C2C12 cells. Specifically, C2C12 cells from a well of a 48-well plate were incubated for one hour with the mRNA/pepMB1 complexes. 18 hours later, luciferase activity was measured (FIG. 6). Untransfected non-lysed C2C12 cells served as negative control while untransfected C2C12 cells lysed with Triton X-100, to release all of the LDH into the culture medium, served as a positive control.

### Results

mRNA mixtures did not induce cytotoxicity, even at the highest molar ratio (MB8-luc2 mRNA:MB8-2Apro mRNA ratio of 9:1). This indicates that the expression of the viral protease does not induce cytotoxicity (FIG. 9).

### Example 8: Luciferase Expression Kinetics Optimized by Co-Transfection of MB8-luc2 and MB8-2Apro mRNA

### Materials and Methods

In the absence of toxicity, the MB8-luc2 and MB8-2Apro mRNAs were next co-transfected into C2C12 cells at different ratios, according to the methods described above. 18 hours later, luciferase activity was measured (FIG. 7). Kinetics were then conducted from 6 hours to 7 days post-transfection, in order to determine if the improvement in luciferase expression was observed only 18 hours post-transfection, according to the methods described above.

### Results

Surprisingly, co-transfection of MB8-luc2 and MB8-2Apro mRNAs increased luciferase expression of MB8-luc2 mRNA by at least 2.5-fold in C2C12 cells at all ratios tested. The best MB8-luc2 mRNA:MB8-2Apro mRNA molar ratio is 465:1, and increases luciferase expression by 3.4-fold.

Kinetics conducted from 6 hours to 7 days post-transfection surprisingly demonstrate that improvement in luciferase expression is observed for at least a week. Indeed, luciferase expression is on average increased by 2.4-fold (FIG. 7).

### Example 9: Effect of RNA Aptamers on the Efficiency of mRNA Transfection in Muscle

In order to improve the internalization of the mRNA molecule according to the invention, different aptamers have been incorporated into the RNA molecule, as detailed below. The effect of these aptamers was then evaluated in vivo, to determine if an improvement in luciferase expression could be observed.

### Materials and Methods

### Aptamer Selection

Aptamers penetrating C2C12 cells were selected. First, double-stranded DNA was generated by hybridizing a 5′ primer followed by extension of a single-stranded DNA from a single-stranded DNA library. Double-stranded DNA thus obtained was then precipitated and purified according to methods well-known to the person skilled in the art.

An RNA aptamer library was then obtained by transcription of the purified fragments using the T7 DuraScribe transcription kit (20 μl/run) followed by RNA purification using a ssDNA and RNA purification kit. Finally, the solution is treated with DNAsel to remove the contaminating DNA. Aptamers are dissolved in 1×DMEM+ITS at 8 μM RNA (1288 μg/5 ml). To select the aptamers, 5 ml of the DMEM/ITS/RNA solution is added to cells previously washed twice with DMEM without antibiotics or serum. Cells are incubated at 37° C. for 1 hour, with the flask containing the mixture being briefly shaken every 15 minutes. The flask containing the cells is then placed on ice and cells are washed 5 times with 15 mL of cold 1×PBS to remove the RNA aptamers which have not penetrated cells. Cells are then lysed with TRIzol (Invitrogen) and total RNA extracted by the phenol-chloroform method.

Endogenous RNAs are digested with RNAse A and remaining RNAs hybridized with 3′ primers and reverse transcribed by the Superscript III enzyme (ThermoFisher) before PCR amplification in the presence of 5′ primer (100 μM), 3′ primer (100 μM), Q5 High Fidelity DNA polymerase (NEB) and the Q5 High-Fidelity Master Mix buffer at a 1× concentration. All of these steps allowing for the obtention of an aptamer RNA library are repeated. Thus, two cycles of selection of RNA aptamers penetrating C2C12 cells are performed.

Two RNA aptamers (B and C) penetrating C2C12 cells were selected and sequenced (SEQ ID NO: 65 and 66, respectively). RNA aptamers B and C were then inserted into the 5′-UTR of the MB8-luc2 mRNA, upstream of xrRNA1, to generate the MB13-luc2 and MB14-luc2 mRNAs, respectively.

### Aptamer Which Strongly Binds the Poly-Histidine Peptide Motif

A second strategy aimed at improving mRNA internalization consisted of inserting another RNA aptamer (aptamer A) capable of strongly binding the poly-histidine peptide motif in the presence of magnesium into the 5′-UTR of the MB8-luc2 mRNA, upstream of xrRNA1. The mRNA incorporating RNA aptamer A was named MB11-luc2. A mouse muscle fiber penetrating peptide, M12 (see Gao et al., 2014), was linked via a spacer (here comprising the glycine and serine amino acids) to the hexahistidine motif. Different spacers are illustrated by way of example in the sequences of

SEQ ID NO: 75
(RRQPPRSISSHPGGGGSGGGGSGGGGSGGGGSGGHHHHHH),
SEQ ID NO: 76
(PQRDTVGGRTTPPSWGPAKAGGGGSGGGGSGGGGHHHHHH),
SEQ ID NO: 77
(GPFHFYQFLFPPVGGGGSGGGGSGGGGSGGGGSGHHHHHH)
SEQ ID NO: 78
(GSPWGLQHHPPRTGGGGSGGGGSGGGGSGGGGSGHHHHHH)
(spacer sequence underlined).

The peptide thus formed was named M12-H6 (SEQ ID NO: 75). The MB11-luc2 mRNA was incubated for 30 minutes at room temperature with the M12-H6 peptide, before being injected into the biceps femoris of mice.

### In Vivo Transfection

The biceps femoris muscle of male BALB/cByJ mice was transfected with 5 μg of MB8-luc2 mRNA, MB13-luc2 mRNA, MB14-luc2 mRNA, or MB11-luc2 mRNA complexed with M12-H6 peptide in the presence or absence of 5 μg of chloroquine. Luciferase activity was measured 16 hours after the injections.

### Results

MB13-luc2 mRNA transfected the muscle just as well as MB8-luc2 mRNA (FIG. 10). Very advantageously, MB14-luc2 mRNA comprising the C aptamer transfected the biceps femoris 2.1 times more efficiently than MB8-luc2 mRNA. RNA aptamer C therefore improved internalization of the mRNA molecule into which it was inserted in the muscle fibers.

Transfection efficiency of MB11-luc2 mRNA was modest (FIG. 10). Surprisingly, however, co-injection of 5 μg of M B11-luc2 mRNA, M12-H6 peptide and 5 μg of chloroquine, improved transfection efficiency by 31-fold as compared to MB11-luc2 mRNA in the presence of the peptide, without chloroquine. In addition, luciferase expression obtained with MB11-luc2 mRNA in the presence of M12-H6 and chloroquine was very advantageously 2.7-fold higher than that obtained with MB8-luc2 mRNA.

Without being bound by theory, one could suppose that the high transfection efficiency of MB11-luc2 RNA complexed with the M12-H6 peptide and in the presence of chloroquine was favored by a slowing of endosome acidification by chloroquine following the penetration of mRNA into cells. Chloroquine could slow hexahistidine protonation at acidic pH and therefore prevent destabilization of the complexes between MB11-luc2 mRNA and the M12-H6 peptide. As a result, mRNA escape from the endosome could be enhanced, by crossing the endosomal membrane with the help of M12-H6, to which it is still complexed, to enter the cytosol where mRNA is then translated.

### Example 10: Intradermal Injection Protocol in Mice

### a—Solution Preparation

A 60 μl solution containing 20 μg of mRNA was prepared for each mouse. For this, demineralized water, 50 mM Hepes (1/8 of Hepes, 7/8 of sodium Hepes), NaCl (160 mM final), MgCl₂, an mRNA, and, optionally, a peptide, were mixed. A 30-minute incubation at room temperature allowed the peptide to bind to the RNA. There was no incubation when mRNA was not complexed to a peptide. mRNA solutions were frozen at −80° C. for conservation until they were injected.

### b—Intradermal Injection and Biopsy Sampling

6-week-old male OF1 mice were used (Charles River). They were shaved three to four days before intradermal injection. Anesthesia was performed using a mask. Induction of anesthesia was achieved with 4% isoflurane (Piramal Heathcare). Anesthesia was maintained at a percentage of isoflurane of 2%. Before injection, previously shaved skin of the back was cleaned with an alcohol wipe. mRNA solutions were slowly thawed at room temperature in order to fill 0.3mm×8 mm U-100 (30 G) insulin syringes (Becton-Dickinson). Three injections of approximately 17 μl of RNA solution were performed in the skin of the shaved back of each mouse. Papules formed before resolving. These were delimited using a permanent marker to allow the area of skin to be biopsied to be identified the next day. Tail tagging was also performed using a marker to distinguish between animals from the same cage.

18 hours after injections, skin biopsies were taken from the injected areas. For this, the mice were anesthetized, then euthanized by cervical dislocation. Biopsies were cut into small pieces using a pair of scissors, to facilitate cell lysis, and introduced into tubes containing 500 μl of 1× lysis buffer (5× Luciferase Cell Culture Lysis (Promega), diluted in water). Each tube was frozen at −20° C. until the next step.

### c-Cell Lysis, Luciferase and Protein Assays

Lysis of the collected tissues was obtained by performing three freeze/thaw cycles: −80° C. for 10 minutes, 3 minutes in a water bath at room temperature and mixing using a vortex mixer for a few seconds. Tubes were then centrifuged at 5000×g for 5 minutes at 20° C. to sediment tissue debris. Supernatant was transferred to another tube.

The luciferase assay was performed using the Luciferase Assay System kit (Promega). 20 μl of each sample was introduced into dedicated tubes for the luminometer (Berthold AutoLumat Plus LB 953). The device injected 100 μl of substrate and measured the amount of light emitted (RLU).

The protein assay was performed using the Pierce 660 nm Protein Assay Kit (Thermo scientific). A calibration range was performed with bovine serum albumin (Thermo scientific) and 1× lysis buffer as diluent. It covered a range of 100 to 500 μg of protein per ml. The blank was obtained using 100 μl of 1× lysis buffer. Lysates were in some cases diluted with 1× lysis buffer. 100 μl of each sample was used for the protein assay. 1.5 ml of reagent was added to the blank and samples. After exactly 5 minutes of incubation at room temperature and in the dark, the absorbance of each sample was measured at 660 nm using a spectrophotometer.

### Example 11: Effect of CPPs on the Efficiency of mRNA Transfection in the Skin

The strategy to improve internalization of naked mRNA by binding a cell penetrating peptide (CPP) to RNA aptamer A is not restricted to muscle, as described in Example 9 above. This strategy has been applied here to the skin of mice using different CPPs.

### Materials and Methods

Three CPPs were used here: CPP1, CPP2 and CPP3 (see Kamada et al., 2007 and Lee et al., 2012). They were connected to hexahistidine by a spacer constituted of glycine and serine to form the peptides CPP1-H6, CPP2-H6 and CPP3-H6 having the sequences of SEQ ID NO: 76, 77, and 78, respectively.

MB11-luc2 mRNA was incubated with increasing amounts of CPP3-H6, CPP1-H6, or CPP2-H6 peptide for 30 minutes in previously optimized buffer comprising 160mM NaCl, 0.7 mM MgCl₂ and 5 mM Hepes. MB11-luc2 mRNA alone and MB8-luc2 mRNA diluted in sterile, ultrapure deionized water, supplemented with 160 mM NaCl, were also injected, and are used here as controls.

An intradermal injection of 5.6 μg of MB8-luc2 mRNA or of MB11-luc2 was performed in OF1 mice according to the protocol detailed in Example 10. Mouse skin was also injected with a mixture of MB11-luc2 mRNA and MB8-luc2 mRNA at a ratio of 1:1, and the same amount of CPP3-H6 as that of the 0.5 molar ratio to determine if the CPP3-H6 peptide can dissociate from RNA aptamer A, present in MB11-luc2 mRNA, after intradermal injection. Luciferase activity was measured 18 hours after injections.

### Results

As expected, injection of MB8-luc2 mRNA resulted in efficient skin transfection. Co-injection of CPP3-H6, 0.7 mM MgCl₂ and 5 mM Hepes with MB8-luc2 mRNA did not significantly affect transfection efficiency (FIG. 11).

The optimal amount of CPP3-H6 peptide, which advantageously increases transcription efficiency by 9.2-fold as compared to MB11-luc2 mRNA alone, corresponds to a molar ratio of 1 CPP3-H6 peptide:2 mRNAs (FIG. 11). In addition, luciferase activity is very advantageously 2.1-fold higher than that obtained with MB8-luc2 mRNA and 19.5-fold higher than that obtained with the conventional MB5-luc2 capped mRNA.

When one out of every two mRNAs does not bind CPP3-H6, transfection drops 12.3-fold (FIG. 11, 2.8 μg of each of the MB8-luc2 and MB11-luc2 mRNAs). This means that the CPP3-H6 peptide can dissociate from RNA aptamer A, present in the MB11-luc2 mRNA, after intradermal injection. If half of the mRNA lacks RNA aptamer A (MB8-luc2 mRNA), CPP3-H6 cannot improve transfection as effectively.

MB11-luc2 mRNA was also incubated with the CPP1-H6 peptide at different molar ratios. The best molar ratio was 1 CPP1-H6 peptide:4 MB11-luc2 mRNAs (FIG. 12). Transfection efficiency was increased 5.4-fold as compared to MB11-luc2 mRNA alone.

CPP1-H6 therefore gave a poorer result than CPP3-H6. This can be explained by the number of copies of the receptor(s) of each CPP present in the plasma membrane of skin cells and the affinity of each CPP for its receptor(s).

Finally, MB11-luc2 mRNA was also incubated with CPP2-H6 at various molar ratios. The best molar ratio was 2 CPP2-H6 peptides:1 MB11-luc2 mRNA (FIG. 13). Transfection efficiency increased 10.6-fold as compared to MB11-luc2 mRNA alone, and is also higher than that obtained with the CPP3-H6 peptide. Luciferase expression is thus 22.8-fold higher than that obtained with conventional MB5-luc2 mRNA.

### Example 12: CPP-H6 Effect on Transfection Efficiency of a capped mRNA in the Skin

### Materials and Methods

In order to evaluate the effect of a CPP (here CPP2-H6) on the transfection efficiency of a capped mRNA, aptamer A was inserted into the 5′-UTR of the conventional capped MB5-luc2 mRNA to generate the capped MB15-luc2 mRNA having the sequence of SEQ ID NO: 70. MB15-luc2 mRNA was incubated with CPP2-H6 for 30 minutes in buffer comprising 0.7 mM MgCl₂ and 5 mM Hepes, as described above in Example 11.

An intradermal injection of 5.6 μg of MB15-luc2 mRNA was performed in OF1 mice according to the protocol detailed in Example 10. Luciferase activity was measured 18 hours after injections.

### Results

In the absence of the CPP2-H6 peptide, luciferase expression obtained with the capped MB15-luc2 mRNA is 1.68-fold lower than that obtained with the capped MB5-luc2 mRNA.

Co-injection of the capped MB15-luc2 mRNA and CPP2-H6 peptide, at a molar ratio of 2 peptides per mRNA, only improved transfection efficiency by 1.48-fold as compared to MB15-luc2 mRNA alone. This indicates that the insertion of the RNA aptamer A into the 5′-UTR of a conventional capped mRNA and the attachment of a CPP-H6 peptide to this RNA aptamer does not improve transfection efficiency as compared to that observed with the capped mRNA, MB11-luc2 (10.6-fold). Regardless of the capped mRNA molecule (with or without aptamer, linked or not to the CPP2-H6 CPP), transfection efficiency remains much lower than that observed for the MB8-luc2 and MB11-luc2 mRNAs. It is therefore highly advantageous to use the mRNA molecules of the invention rather than capped mRNA molecules.

### Example 13: Effect of 5′-SL on Luciferase Expression In Vivo in the Skin

### Materials and Methods

The following mRNAs: MB8-luc2, MB11-luc2, MB17-luc2, MB18-luc2, and capped MB5-luc2 were injected into the skin and luciferase activity measured 18 hours after injection, according to the protocol detailed in Example 10 above.

### Results

As illustrated in FIG. 15, the mRNA according to the invention comprising at least one xrRNA sequence and an IRES sequence (MB11-luc2) increases luciferase expression in the skin by approximately 2-fold as compared to the capped mRNA (MB5-luc2). Very surprisingly, the addition of a stem-loop (here the 5′-SL having the sequence of SEQ ID NO: 87) increases luciferase expression in the skin by about an additional 4.3-fold over the sole xrRNA and IRES sequences (MB11-luc2), when the stem-loop is located five nucleotides upstream of the xrRNA1 sequence (MB8-luc2 and MB18-luc2).

However, when the stem-loop is located approximately 70 nucleotides upstream of the xrRNA sequence, the stem-loop does not improve efficiency beyond what is already observed in the absence of the stem-loop. Indeed, translation efficiency is similar for the MB11-luc2 and MB17-luc2 mRNAs. The placement of aptamer A between the xrRNA sequences and the IRES sequence (MB18-luc2) increases translation efficiency even more as compared to the MB11-luc2 and MB17-luc2 mRNAs in a very surprising way. MB8-luc2 and MB18-luc2 mRNAs thus have a similar translation efficiency.

### Conclusion

An mRNA molecule according to the invention has a cost of synthesis which is reduced by approximately 30-fold as compared to a capped mRNA molecule, while having a yield in protein expression that is increase by at least 2-fold, preferably about 10-fold. In addition, the mRNA of the invention is at least as stable as a capped mRNA, and its production by in vitro transcription is simplified by the absence of a cap molecule or an analog thereof. An mRNA of the invention coding the 2A protease of HRV2 makes it possible to increase the translation of an mRNA coding a protein of interest when these two molecules are co-transfected. In addition, the efficiency of transfection in a tissue, such as muscle or skin, can be improved, e.g., by inserting into the 5′-UTR region of the mRNA molecule according to the invention an RNA aptamer directly penetrating cells, a CPP (attached to an RNA aptamer as described above), and/or by the addition of a stem-loop at the 5′ end of the 5′-UTR region upstream of the xrRNA sequence(s).

### REFERENCES

Ausubel et al., 2001, Current Protocols in Molecular Biology, Wiley & Sons, Hoboken, N.J., USA.

Beckert and Masquida, 2011, Methods Mol Biol. 703: 29-41.

Borman et al., 1995, Nucl. Acids Res. 23(18): 3656-3663.

Chapman et al., 2014, eLife, 3: e01892.

Contreas et al., 1982, Nucl. Acids Res. 10: 6353-6363.

Cowling, 2010, Biochem. J. 425: 295-302.

Dunn et al., 2017, Nat Rev Chem. 1. s41570-017. 10.1038/s41570-017-0076.

Gao et al., 2014, Molecular Therapy 22(7): 1333-41.

Goldstaub et al., 2000, Mol Cell Biol, 20: 1271-1277.

Kamada et al., 2007, Biol. Pharm. Bull. 30(2): 218-23.

Kieft et al., 2015, RNA Biology, 12(11): 1169-77.

Lee et al., 2012, Biotechnol. 7: 387-96.

Martinez Salas et al., 2013, Int J Mol Sci. November; 14(11): 21705-21726.

Martin et al., 1975, JBC, 250: 9322-9329.

Mokrejš et al., 2010, Nucl. Acids Res. 38 (suppl 1): D131-D13.

Needleman and Wunsch, 1970, J Mol Biol. 48(3):443-53.

Pasquinelli et al., 1995, RNA, 1: 957-967.

Poillot and De Waard, 2011, Med Sci (Paris). 27(5):527-34.

Rogé and Betton, 2005, Microb Cell Fact. 4: 18.

Tsuji S. et al., 2013, PLoS ONE 8(12): e83108.

1. A messenger ribonucleic acid (mRNA) molecule lacking a cap molecule comprising from 5′ to 3′:
a 5′-UTR region comprising at least one copy of a consensus sequence GUCAGRYC(N₇₋₁₉)GCCA(N₁₂₋₁₉)UGCNRYCUG (xrRNA);
a copy of an internal ribosome entry site (IRES) RNA sequence; and
at least one open reading frame.

2. The mRNA molecule according to claim 1, comprising two copies of xrRNA.

3. The mRNA molecule according to claim 1, comprising at least one sequence having the SEQ ID NO of any one of SEQ ID NO: 1 to 44.

4. The mRNA molecule according to claim 1, comprising the IRES sequence of encephalomyocarditis virus.

5. The mRNA molecule according to claim 1, comprising a stem-loop, wherein the stem-loop is located at the 5′ end of the 5′-UTR region.

6. The mRNA molecule according to claim 1, comprising at least one aptamer selected from aptamer A having the sequence of SEQ ID NO: 64 and aptamer C having the sequence of SEQ ID NO: 66.

7. The mRNA molecule comprising aptamer A according to claim 6, wherein the mRNA molecule is bound to a cell-penetrating peptide (CPP) fused to a poly-histidine tag.

8. The mRNA molecule according claim 1, wherein an open reading frame codes for the 2Apro protein of the HRV2 virus.

9. A deoxyribonucleic acid (DNA) molecule comprising a sequence coding the mRNA according to claim 1.

10. A vector comprising the mRNA molecule according to claim 1.

11. An in vitro method for producing at least one mRNA comprising contacting the DNA molecule according to claim 9 with at least one RNA polymerase.

12. The method according to claim 11, comprising a step of purifying the mRNA.

13. A pharmaceutically acceptable composition comprising the mRNA according to claim 1 and a physiologically acceptable excipient and/or adjuvant.

14. A method of enhancing or inducing an immune response to a polypeptide in a subject comprising administering the composition according to claim 13.

15. The composition according to claim 13, comprising a second mRNA molecule lacking a cap molecule comprising from 5′ to 3′:
a 5′-UTR region comprising at least one copy of a consensus sequence GUCAGRYC(N₇₋₁₉)GCCA(N₁₂₋₁₉)UGCNRYCUG (xrRNA);
a copy of an internal ribosome entry site (IRES) RNA sequence; and
at least one open reading frame,
wherein in the second mRNA molecule, an open reading frame codes the 2Apro protein.

16. The mRNA molecule according to claim 3, comprising SEQ ID NO: 11 and SEQ ID NO: 26.

17. The mRNA molecule according to claim 5, wherein the stem-loop has the sequence of SEQ ID NO: 87.

18. The mRNA molecule comprising aptamer A according to claim 7, wherein the CPP is selected from M12-H6 having the sequence of SEQ ID NO: 75, CPP1-H6 having the sequence of SEQ ID NO: 76, CPP2-H6 having the sequence of SEQ ID NO: 77, and CPP3-H6 having the sequence of SEQ ID NO: 78.

19. The DNA molecule according to claim 9, comprising
a promoter recognized by the T7 RNA polymerase, the promoter comprising a sequence represented by SEQ ID NO: 46;
a 5′-UTR region comprising a sequence represented by SEQ ID NO: 50, 71, 72, 73, 85, or 86;
an open reading frame; and
a 3′-UTR region comprising a sequence selected from the sequences represented by SEQ ID NOs: 53, 54, 55, and 56.

20. A vector comprising the DNA molecule according to claim 9.


SEQUENCE LISTING (89 sequences)

SEQ ID NO: 1 - RNA, 45 nt, Dengue virus type 1
gucaggccggauuaagccauagcacgguaagagcuaugcugccug

SEQ ID NO: 2 - RNA, 45 nt, Dengue virus type 2
gucaggccggauuaagccauaguacggaaaaaacuaugcuaccug

SEQ ID NO: 3 - RNA, 45 nt, Dengue virus type 3
gucaggccaccuuaagccacaguacggaagaagcugugcugccug

SEQ ID NO: 4 - RNA, 43 nt, Kokobera Virus
gucaggccugaaaagccaccugauccggugaaggugcugccug

SEQ ID NO: 5 - RNA, 47 nt, Zika Virus
gucaggccugcuagucagccacaguuuggggaaagcugugcagccug

SEQ ID NO: 6 - RNA, 50 nt, Saint Louis Encephalitis Virus
gucaggccaaucaguuuugccaccggaugucagguaaacggugcugucug

SEQ ID NO: 7 - RNA, 50 nt, Japanese Encephalitis Virus
gucaggccagcaaaagcugccaccggauacuggguagacggugcugucug

SEQ ID NO: 8 - RNA, 50 nt, Ntaya Virus
gucaggccaggcaaugccugccaccggaaguuggaugacggugcugucug

SEQ ID NO: 9 - RNA, 50 nt, Usutu Virus
gucaggccagggcaaccugccaccggaaguugaguagacggugcugccug

SEQ ID NO: 10 - RNA, 52 nt, Murray Valley Encephalitis Virus
gucaggccagccgguuaggcugccaccgaagguugguagacggugcugccug

SEQ ID NO: 11 - RNA, 51 nt, West Nile Virus
gucaggccagauuaaugcugccaccggaaguugaguagacggugcugccug

SEQ ID NO: 12 - RNA, 45 nt, Iguape Virus
gucaggccggaaacgccaccggauggucguaaacggugcggccug

SEQ ID NO: 13 - RNA, 52 nt, Bagaza Virus
gucaggccggacuacguguccgccaccggauguuggaugacggugcugccug

SEQ ID NO: 14 - RNA, 52 nt, Israel Turkey Meningoencephalomyelitis Virus
gucaggccggauuauauguccgccaccggauguuggaugacggugcugccug

SEQ ID NO: 15 - RNA, 42 nt, New Mapoon Virus
gucaggccgaauagccaucugauccggugaagaugcugccug

SEQ ID NO: 16 - RNA, 41 nt, Dengue virus type 1
gucaggccgaaagccacgguucgagcaagccgugcugccug

SEQ ID NO: 17 - RNA, 46 nt, Dengue virus type 2
gucaggccaucauaaaugccauagcuugaguaaacuaugcagccug

SEQ ID NO: 18 - RNA, 43 nt, Dengue virus type 4
gucaggccacuugugccacgguuugagcaaaccgugcugccug

SEQ ID NO: 19 - RNA, 37 nt, Kokobera Virus
gucagauccgaaaggccaccaguuuggugcagaacug

SEQ ID NO: 20 - RNA, 44 nt, Zika Virus
gucaggccgagaacgccauggcacggaagaagccaugcugccug

SEQ ID NO: 21 - RNA, 46 nt, Saint Louis Encephalitis Virus
gucagaccagaaaugccaccugaaagcaugcuaaaggugcugucug

SEQ ID NO: 22 - RNA, 44 nt, Japanese Encephalitis Virus
gucaggccacaaauuugugccaccccgcuagggggugcggccug

SEQ ID NO: 23 - RNA, 43 nt, Ntaya Virus
gucaggccguagguuuuacgccacuagcaugcagugcugccug

SEQ ID NO: 24 - RNA, 41 nt, Usutu Virus
gucaggccgcaaagcgccacuucgccaaggagugcagccug

SEQ ID NO: 25 - RNA, 42 nt, Murray Valley Encephalitis Virus
gucagaucgcgaaagcgccacuucgccgaggagugcaaucug

SEQ ID NO: 26 - RNA, 45 nt, West Nile Virus
gucagaccacacuuuaaugugccacucugcggagagugcagucug

SEQ ID NO: 27 - RNA, 43 nt, Iguape Virus
gucaggccgaaagccaccacaagcgguacaguggugcugccug

SEQ ID NO: 28 - RNA, 43 nt, Bagaza Virus
gucaggccacagguuuugugccacuagcaugcagugcugccug

SEQ ID NO: 29 - RNA, 43 nt, Israel Turkey Meningoencephalomyelitis Virus; identical to SEQ ID NO: 28

SEQ ID NO: 30 - RNA, 48 nt, New Mapoon Virus
gucagaccacuuagugccaccaguaugaugauaagcuggugcugucug

SEQ ID NO: 31 - RNA, 50 nt, Wesselbron Virus
gucagcccaucauuugaugccauggcuaagcugugaggccaugcuggcug

SEQ ID NO: 32 - RNA, 49 nt, Ilheus Virus
gucaggccauggaaacaugccacccaaagcuuguagagggugcagccug

SEQ ID NO: 33 - RNA, 51 nt, Sepik Virus
gucagcccgucauaaugacgccauggcuaagcugugaggccaugcuggcug

SEQ ID NO: 34 - RNA, 45 nt, Bussuquara Virus
gucaggccagaaaugccaccggauaaagguagacggugcugccug

SEQ ID NO: 35 - RNA, 50 nt, Tembusu Virus
gucaggccagggaaucccugccaccggauguuggaugacggugcugucug

SEQ ID NO: 36 - RNA, 44 nt, Chaoyang Virus
gucaggccuaaaugccaccggaugauaguagacggugcugccug

SEQ ID NO: 37 - RNA, 50 nt, Kedougou Virus
gucaggccacucgugagagugccacaguacgguaaagacugugcggccug

SEQ ID NO: 38 - RNA, 53 nt, Yokose Virus
gucaggccaagauugagaaaaucuugccacagcuuggcagacugugcagccug

SEQ ID NO: 39 - RNA, 51 nt, Donggang Virus
gucaggccucacgaaugugagccaccggaugggacuagacggugcugccug

SEQ ID NO: 40 - RNA, 59 nt, Yellow Fever Virus
gucagcccagaaccccacacgaguuuugccacugcuaagcugugaggcagugcaggcug

SEQ ID NO: 41 - RNA, 48 nt, Rocio Virus
gucaggccguccuuggacgccacccaaagcaugggagggugcugccug

SEQ ID NO: 42 - RNA, 49 nt, Alfuy Virus
gucaggccagugaaaacugccaccggauguugguagacggugcugccug

SEQ ID NO: 43 - RNA, 43 nt, Stratford Virus
gucaggccugaaaagccaucugauccggugaagaugcugccug

SEQ ID NO: 44 - RNA, 50 nt, Duck Eggdrop Syndrome Virus; identical to SEQ ID NO: 35

SEQ ID NO: 45 - DNA, 547 bp, Encephalomyocarditis virus
tactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 46 - DNA, 17 bp, Bacteriophage T7
taatacgactcactata

SEQ ID NO: 47 - RNA, 83 nt, Artificial
  Spacer 1 between xrRNA sequences in RNA from pMB8-luc2 and pMB9-luc2
gaccaaagcugcgaggugauccacguaagcccucagaaccgucucggaaggaggaccccacgugcuuuagccucaaagcccag

SEQ ID NO: 48 - RNA, 17 nt, Artificial
  Spacer 2 between xrRNA2 and IRES sequences in RNA from pMB8-luc2 and pMB9-luc2
uaacaaaggcaaaacau

SEQ ID NO: 49 - DNA, 63 bp, Artificial
  5-UTR pMB5-luc2 (without T7 promoter)
gggaagcttaagtgttctttttgcagaagctcagaataaacgctcaactttggcagatctacc

SEQ ID NO: 50 - DNA, 818 bp, Artificial
  5-UTR of pMB8-luc2 and pMB9-luc2
gggaagcttaagcttcccaaaaaagtcaggccagattaatgctgccaccggaagttgagtagacggtgctgcctgcggctcaaccccaggaggactgggtgaccaaagctgcgaggtgatccacgtaagccctcagaaccgtctcggaaggaggaccccacgtgctttagcctcaaagcccagtgtcagaccacactttaatgtgccactctgcggagagtgcagtctgcgatagtgccccaggtggactgggttaacaaaggcaaaacattactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 51 - DNA, 583 bp, Artificial
  5-UTR of pMB7-luc2 (without T7 promoter)
gggaagcttaagcttcccttaacaaaggcaaaacattactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 52 - DNA, 44 bp, Artificial
  3′-UTR of pMB5-luc2, pMB7-luc2 et pMB8-luc2 without poly A tail or restriction site
ttctagaatgtccgaatggttgacacttgatctcggcaacgcat

SEQ ID NO: 53 - DNA, 109 bp, Artificial
  3′-UTR of pMB5-luc2, pMB7-luc2 et pMB8-luc2 without restriction site
ttctagaatgtccgaatggttgacacttgatctcggcaacgcataaaaaaaaaaaaaaaaaaaaaaaaaaaaaaaaaaaaaaaaaaaaaaaaaaaaaaaaaaaaaaaaa

SEQ ID NO: 54 - DNA, 113 bp, Artificial
  Complete 3′-UTR of pMB5-luc2, pMB7-luc2 et pMB8-luc2 w SspI restruction site
ttctagaatgtccgaatggttgacacttgatctcggcaacgcataaaaaaaaaaaaaaaaaaaaaaaaaaaaaaaaaaaaaaaaaaaaaaaaaaaaaaaaaaaaaaaaatatt

SEQ ID NO: 55 - DNA, 625 bp, Kunjin virus
aatactttgttaattgtaaataaatattgttattatgtgtagaagtttagctttataatagtgtttagtgtgtttagagttagaaaaattttagtgaggaagtcaggccggaaaattcccgccaccggaagttgagtagacggtgctgcctgcgactcaaccccaggaggactgggtgaacaaagctgcgaagtgatccatgtaagccctcagaaccgtctcggaaagaggaccccacatgttgtagcttcaaggcccaatgtcagaccacgccatggcgtgccactctgcggagagtgcagtctgcgacagtgccccaggaggactgggtgaacaaaggcgaatcaacgtcccacgcggccctagctctggcaatggtgttaaccagagtgaaaggactagaggttagaggagaccccgcgttctgaagtgcacggcccagcctggctgaagctgtaggtcaggggaaggactagaggttagtggagaccccgtgccgcaaaacaccacaacaacacagcatattgacacctgggatagactaggagatcttctgctctgcacaaccagccacacggcacagtgcgccgacaatggtggctggtggtgcgagaacacaggatct

SEQ ID NO: 56 - DNA, 718 bp, Artificial
  3-UTR (Kunjin virus) - Eco53kI (pMB9-luc2)
ttctagaatactttgttaattgtaaataaatattgttattatgtgtagaagtttagctttataatagtgtttagtgtgtttagagttagaaaaattttagtgaggaagtcaggccggaaaattcccgccaccggaagttgagtagacggtgctgcctgcgactcaaccccaggaggactgggtgaacaaagctgcgaagtgatccatgtaagccctcagaaccgtctcggaaagaggaccccacatgttgtagcttcaaggcccaatgtcagaccacgccatggcgtgccactctgcggagagtgcagtctgcgacagtgccccaggaggactgggtgaacaaaggcgaatcaacgtcccacgcggccctagctctggcaatggtgttaaccagagtgaaaggactagaggttagaggagaccccgcgttctgaagtgcacggcccagcctggctgaagctgtaggtcaggggaaggactagaggttagtggagaccccgtgccgcaaaacaccacaacaacacagcatattgacacctgggatagactaggagatcttctgctctgcacaaccagccacacggcacagtgcgccgacaatggtggctggtggtgcgagaacacaggatctgggtcggcatggcatctccacctcctcgcggtccgacctgggcatccgaaggaggacgcacgtccactcggatggctaagggagctc

SEQ ID NO: 57 - RNA, 63 nt, Artificial
  5′-UTR of RNA from MB5-luc2
gggaagcuuaaguguucuuuuugcagaagcucagaauaaacgcucaacuuuggcagaucuacc

SEQ ID NO: 58 - RNA, 583 nt, Artificial
  5′-UTR of RNA from MB7-luc2
gggaagcuuaagcuucccuuaacaaaggcaaaacauuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgaugauaau

SEQ ID NO: 59 - RNA, 818 nt, Artificial
  5-UTR of pMB8-luc2 and pMB9-luc2
gggaagcuuaagcuucccaaaaaagucaggccagauuaaugcugccaccggaaguugaguagacggugcugccugcggcucaaccccaggaggacugggugaccaaagcugcgaggugauccacguaagcccucagaaccgucucggaaggaggaccccacgugcuuuagccucaaagcccagugucagaccacacuuuaaugugccacucugcggagagugcagucugcgauagugccccagguggacuggguuaacaaaggcaaaacauuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgaugauaau

SEQ ID NO: 60 - RNA, 110 nt, Artificial
  3′-UTR of RNA from MB5-luc2, MB7-luc2, MB8-luc2
uucuagaauguccgaaugguugacacuugaucucggcaacgcauaaaaaaaaaaaaaaaaaaaaaaaaaaaaaaaaaaaaaaaaaaaaaaaaaaaaaaaaaaaaaaaaau

SEQ ID NO: 61 - RNA, 715 nt, Artificial
  3′-UTR of RNA from MB9-luc2
uucuagaauacuuuguuaauuguaaauaaauauuguuauuauguguagaaguuuagcuuuauaauaguguuuaguguguuuagaguuagaaaaauuuuagugaggaagucaggccggaaaauucccgccaccggaaguugaguagacggugcugccugcgacucaaccccaggaggacugggugaacaaagcugcgaagugauccauguaagcccucagaaccgucucggaaagaggaccccacauguuguagcuucaaggcccaaugucagaccacgccauggcgugccacucugcggagagugcagucugcgacagugccccaggaggacugggugaacaaaggcgaaucaacgucccacgcggcccuagcucuggcaaugguguuaaccagagugaaaggacuagagguuagaggagaccccgcguucugaagugcacggcccagccuggcugaagcuguaggucaggggaaggacuagagguuaguggagaccccgugccgcaaaacaccacaacaacacagcauauugacaccugggauagacuaggagaucuucugcucugcacaaccagccacacggcacagugcgccgacaaugguggcugguggugcgagaacacaggaucugggucggcauggcaucuccaccuccucgcgguccgaccugggcauccgaaggaggacgcacguccacucggauggcuaagggag

SEQ ID NO: 62 - DNA, 1656 bp, Artificial
  luc2
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaataa

SEQ ID NO: 63 - RNA, 1656 nt, Artificial
  luc2
auggaagaugccaaaaacauuaagaagggcccagcgccauucuacccacucgaagacgggaccgccggcgagcagcugcacaaagccaugaagcgcuacgcccuggugcccggcaccaucgccuuuaccgacgcacauaucgagguggacauuaccuacgccgaguacuucgagaugagcguucggcuggcagaagcuaugaagcgcuaugggcugaauacaaaccaucggaucguggugugcagcgagaauagcuugcaguucuucaugcccguguugggugcccuguucaucgguguggcuguggccccagcuaacgacaucuacaacgagcgcgagcugcugaacagcaugggcaucagccagcccaccgucguauucgugagcaagaaagggcugcaaaagauccucaacgugcaaaagaagcuaccgaucauacaaaagaucaucaucauggauagcaagaccgacuaccagggcuuccaaagcauguacaccuucgugacuucccauuugccacccggcuucaacgaguacgacuucgugcccgagagcuucgaccgggacaaaaccaucgcccugaucaugaacaguaguggcaguaccggauugcccaagggcguagcccuaccgcaccgcaccgcuuguguccgauucagucaugcccgcgaccccaucuucggcaaccagaucauccccgacaccgcuauccucagcguggugccauuucaccacggcuucggcauguucaccacgcugggcuacuugaucugcggcuuucgggucgugcucauguaccgcuucgaggaggagcuauucuugcgcagcuugcaagacuauaagauucaaucugcccugcuggugcccacacuauuuagcuucuucgcuaagagcacucucaucgacaaguacgaccuaagcaacuugcacgagaucgccagcggcggggcgccgcucagcaaggagguaggugaggccguggccaaacgcuuccaccuaccaggcauccgccagggcuacggccugacagaaacaaccagcgccauucugaucacccccgaaggggacgacaagccuggcgcaguaggcaagguggugcccuucuucgaggcuaaggugguggacuuggacaccgguaagacacugggugugaaccagcgcggcgagcugugcguccguggccccaugaucaugagcggcuacguuaacaaccccgaggcuacaaacgcucucaucgacaaggacggcuggcugcacagcggcgacaucgccuacugggacgaggacgagcacuucuucaucguggaccggcugaagagccugaucaaauacaagggcuaccagguagccccagccgaacuggagagcauccugcugcaacaccccaacaucuucgacgccggggucgccggccugcccgacgacgaugccggcgagcugcccgccgcagucgucgugcuggaacacgguaaaaccaugaccgagaaggagaucguggacuauguggccagccagguuacaaccgccaagaagcugcgcggugguguuguguucguggacgaggugccuaaaggacugaccggcaaguuggacgcccgcaagauccgcgagauucucauuaaggccaagaagggcggcaagaucgccguguaauaa

SEQ ID NO: 64 - RNA, 33 nt, Artificial
  aptamer A
gguauauuggcgccuucguggaaugucagugcc

SEQ ID NO: 65 - RNA, 32 nt, Artificial
  aptamer B
gggaggacgaugcggacagacgacucgcccga

SEQ ID NO: 66 - RNA, 36 nt, Artificial
  aptamer C
gggaggacgaugcggacgugcagacgacucgcccga

SEQ ID NO: 67 - RNA, 869 nt, Artificial
  5′-UTR of RNA from MB11-luc2
gggaagcuuaaguggugagguauauuggcgccuucguggaaugucagugccucaccagcaugccuucccaaaaaagucaggccagauuaaugcugccaccggaaguugaguagacggugcugccugcggcucaaccccaggaggacugggugaccaaagcugcgaggugauccacguaagcccucagaaccgucucggaaggaggaccccacgugcuuuagccucaaagcccagugucagaccacacuuuaaugugccacucugcggagagugcagucugcgauagugccccagguggacuggguuaacaaaggcaaaacauuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgaugauaau

SEQ ID NO: 68 - RNA, 874 nt, Artificial
  5′-UTR of RNA from MB13-luc2
gggaagcuuaagcuucccaaaaaagggaggacgaugcggacagacgacucgcccgaaaaaaaucuagagcaugcaaaaaagucaggccagauuaaugcugccaccggaaguugaguagacggugcugccugcggcucaaccccaggaggacugggugaccaaagcugcgaggugauccacguaagcccucagaaccgucucggaaggaggaccccacgugcuuuagccucaaagcccagugucagaccacacuuuaaugugccacucugcggagagugcagucugcgauagugccccagguggacuggguuaacaaaggcaaaacauuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgaugauaau

SEQ ID NO: 69 - RNA, 878 nt, Artificial
  5′-UTR of RNA from MB14-luc2
gggaagcuuaagcuucccaaaaaagggaggacgaugcggacgugcagacgacucgcccgaaaaaaaucuagagcaugcaaaaaagucaggccagauuaaugcugccaccggaaguugaguagacggugcugccugcggcucaaccccaggaggacugggugaccaaagcugcgaggugauccacguaagcccucagaaccgucucggaaggaggaccccacgugcuuuagccucaaagcccagugucagaccacacuuuaaugugccacucugcggagagugcagucugcgauagugccccagguggacuggguuaacaaaggcaaaacauuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgaugauaau

SEQ ID NO: 70 - RNA, 114 nt, Artificial
  5′-UTR of RNA from capped MB15-luc2
gggaagcuuaaguguucuuuuugcagaagcucagaauuggugagguauauuggcgccuucguggaaugucagugccucaccagcaugcaaacgcucaacuuuggcagaucuacc

SEQ ID NO: 71 - DNA, 869 bp, Artificial
  5′-UTR of MB11-luc2
gggaagcttaagtggtgaggtatattggcgccttcgtggaatgtcagtgcctcaccagcatgccttcccaaaaaagtcaggccagattaatgctgccaccggaagttgagtagacggtgctgcctgcggctcaaccccaggaggactgggtgaccaaagctgcgaggtgatccacgtaagccctcagaaccgtctcggaaggaggaccccacgtgctttagcctcaaagcccagtgtcagaccacactttaatgtgccactctgcggagagtgcagtctgcgatagtgccccaggtggactgggttaacaaaggcaaaacattactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 72 - DNA, 874 bp, Artificial
  5′-UTR from MB13-luc2
gggaagcttaagcttcccaaaaaagggaggacgatgcggacagacgactcgcccgaaaaaaatctagagcatgcaaaaaagtcaggccagattaatgctgccaccggaagttgagtagacggtgctgcctgcggctcaaccccaggaggactgggtgaccaaagctgcgaggtgatccacgtaagccctcagaaccgtctcggaaggaggaccccacgtgctttagcctcaaagcccagtgtcagaccacactttaatgtgccactctgcggagagtgcagtctgcgatagtgccccaggtggactgggttaacaaaggcaaaacattactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 73 - DNA, 878 bp, Artificial
  5′-UTR from MB14-luc2
gggaagcttaagcttcccaaaaaagggaggacgatgcggacgtgcagacgactcgcccgaaaaaaatctagagcatgcaaaaaagtcaggccagattaatgctgccaccggaagttgagtagacggtgctgcctgcggctcaaccccaggaggactgggtgaccaaagctgcgaggtgatccacgtaagccctcagaaccgtctcggaaggaggaccccacgtgctttagcctcaaagcccagtgtcagaccacactttaatgtgccactctgcggagagtgcagtctgcgatagtgccccaggtggactgggttaacaaaggcaaaacattactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 74 - DNA, 114 bp, Artificial
  5′-UTR from capped MB15-luc2
gggaagcttaagtgttctttttgcagaagctcagaattggtgaggtatattggcgccttcgtggaatgtcagtgcctcaccagcatgcaaacgctcaactttggcagatctacc

SEQ ID NO: 75 - protein, 40 aa, Artificial
  M12-H6
RRQPPRSISSHPGGGGSGGGGSGGGGSGGGGSGGHHHHHH

SEQ ID NO: 76 - protein, 40 aa, Artificial
  CPP1-H6
PQRDTVGGRTTPPSWGPAKAGGGGSGGGGSGGGGHHHHHH

SEQ ID NO: 77 - protein, 40 aa, Artificial
  CPP2-H6
GPFHFYQFLFPPVGGGGSGGGGSGGGGSGGGGSGHHHHHH

SEQ ID NO: 78 - protein, 40 aa, Artificial
  CPP3-H6
GSPWGLQHHPPRTGGGGSGGGGSGGGGSGGGGSGHHHHHH

SEQ ID NO: 79 - RNA, 462 nt, Human rhinovirus 2
auggugacaaggcccaucaucacaacagccggcccuuccgauauguacgugcacgugggcaaccugaucuacaggaaccugcaccuguucaacagcgagaugcacgagagcauccuggugagcuacagcagcgaucugaucaucuacagaaccaacacagugggcgacgauuacauccccagcugugacugcacacaggccaccuacuacugcaagcacaagaacagauacuucccuaucacagugacaagccacgauugguacgagauccaggagagcgaguacuacccuaagcacauccaguacaaccugcugaucggcgagggcccuugcgagcccggagacuguggcggaaagcugcuguguaagcacggcgugaucggcaucgugaccgccggcggagauaaccacguggccuucaucgaccugaggcacuuccacugcgccgaggagcaguaauaa

SEQ ID NO: 80 - RNA, 1390 nt, Artificial
  MB8-2Apro mRNA
gggaagcuuaagcuucccaaaaaagucaggccagauuaaugcugccaccggaaguugaguagacggugcugccugcggcucaaccccaggaggacugggugaccaaagcugcgaggugauccacguaagcccucagaaccgucucggaaggaggaccccacgugcuuuagccucaaagcccagugucagaccacacuuuaaugugccacucugcggagagugcagucugcgauagugccccagguggacuggguuaacaaaggcaaaacauuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgaugauaauauggugacaaggcccaucaucacaacagccggcccuuccgauauguacgugcacgugggcaaccugaucuacaggaaccugcaccuguucaacagcgagaugcacgagagcauccuggugagcuacagcagcgaucugaucaucuacagaaccaacacagugggcgacgauuacauccccagcugugacugcacacaggccaccuacuacugcaagcacaagaacagauacuucccuaucacagugacaagccacgauugguacgagauccaggagagcgaguacuacccuaagcacauccaguacaaccugcugaucggcgagggcccuugcgagcccggagacuguggcggaaagcugcuguguaagcacggcgugaucggcaucgugaccgccggcggagauaaccacguggccuucaucgaccugaggcacuuccacugcgccgaggagcaguaauaauucuagaauguccgaaugguugacacuugaucucggcaacgcauaaaaaaaaaaaaaaaaaaaaaaaaaaaaaaaaaaaaaaaaaaaaaaaaaaaaaaaaaaaaaaaaau

SEQ ID NO: 81 - protein, 143 aa, Human rhinovirus 2
MGPSDMYVHVGNLIYRNLHLFNSEMHESILVSYSSDLIIYRTNTVGDDYIPSCDCTQATYYCKHKNRYFPITVTSHDWYEIQESEYYPKHIQYNLLIGEGPCEPGDCGGKLLCKHGVIGIVTAGGDNHVAFIDLRHFHCAEEQ

SEQ ID NO: 82 - RNA, 547 nt, Encephalomyocarditis virus
uacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgaugauaau

SEQ ID NO: 83 - RNA, 885 nt, Artificial
  5-UTR of RNA from MB17-luc2
gggaagcuuaagcuucccacaaaaaaucgguauauuggcgccuucguggaaugucagugccucaacaucuagagcaugccuucccaaaaaagucaggccagauuaaugcugccaccggaaguugaguagacggugcugccugcggcucaaccccaggaggacugggugaccaaagcugcgaggugauccacguaagcccucagaaccgucucggaaggaggaccccacgugcuuuagccucaaagcccagugucagaccacacuuuaaugugccacucugcggagagugcagucugcgauagugccccagguggacuggguuaacaaaggcaaaacauuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgaugauaau

SEQ ID NO: 84 - RNA, 873 nt, Artificial
  5-UTR of RNA from MB18-luc2
gggaagcuuaagcuucccaaaaaagucaggccagauuaaugcugccaccggaaguugaguagacggugcugccugcggcucaaccccaggaggacugggugaccaaagcugcgaggugauccacguaagcccucagaaccgucucggaaggaggaccccacgugcuuuagccucaaagcccagugucagaccacacuuuaaugugccacucugcggagagugcagucugcgauagugccccagguggacuggguuaacaaaggcaaaacauggugagguauauuggcgccuucguggaaugucagugccucaccuaagcaugccguuacuggccgaagccgcuuggaauaaggccggugugcguuugucuauauguuauuuuccaccauauugccgucuuuuggcaaugugagggcccggaaaccuggcccugucuucuugacgagcauuccuaggggucuuuccccucucgccaaaggaaugcaaggucuguugaaugucgugaaggaagcaguuccucuggaagcuucuugaagacaaacaacgucuguagcgacccuuugcaggcagcggaaccccccaccuggcgacaggugccucugcggccaaaagccacguguauaagauacaccugcaaaggcggcacaaccccagugccacguugugaguuggauaguuguggaaagagucaaauggcucuccucaagcguauucaacaaggggcugaaggaugcccagaagguaccccauuguaugggaucugaucuggggccucggugcacaugcuuuacauguguuuagucgagguuaaaaaacgucuaggccccccgaaccacggggacgugguuuuccuuugaaaaacacgaugauaau

SEQ ID NO: 85 - DNA, 885 bp, Artificial
  5-UTR of MB17-luc2
gggaagcttaagcttcccacaaaaaatcggtatattggcgccttcgtggaatgtcagtgcctcaacatctagagcatgccttcccaaaaaagtcaggccagattaatgctgccaccggaagttgagtagacggtgctgcctgcggctcaaccccaggaggactgggtgaccaaagctgcgaggtgatccacgtaagccctcagaaccgtctcggaaggaggaccccacgtgctttagcctcaaagcccagtgtcagaccacactttaatgtgccactctgcggagagtgcagtctgcgatagtgccccaggtggactgggttaacaaaggcaaaacattactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 86 - DNA, 873 bp, Artificial
  5-UTR of MB18-luc2
gggaagcttaagcttcccaaaaaagtcaggccagattaatgctgccaccggaagttgagtagacggtgctgcctgcggctcaaccccaggaggactgggtgaccaaagctgcgaggtgatccacgtaagccctcagaaccgtctcggaaggaggaccccacgtgctttagcctcaaagcccagtgtcagaccacactttaatgtgccactctgcggagagtgcagtctgcgatagtgccccaggtggactgggttaacaaaggcaaaacatggtgaggtatattggcgccttcgtggaatgtcagtgcctcacctaagcatgccgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 87 - RNA, 18 nt, Artificial
  5′-SL
gggaagcuuaagcuuccc

SEQ ID NO: 88 - RNA, 10 nt, Artificial Sequence
  pepMB1
crrrrrrrrc

SEQ ID NO: 89 - RNA, 23 nt, Artificial Sequence
  Consensus sequence wherein the ′N′ at position 9 corresponds to 7 to 19 nucleotides of any sequence and wherein the ′N′ at position 14 corresponds to 12 to 19 nucleotides of any sequence
  misc_feature (9)..(9): n is a, c, g, or u
  misc_feature (14)..(14): n is a, c, g, or u
  misc_feature (18)..(18): n is a, c, g, or u
gucagrycngccanugcnrycug